(19) United States
(12) Patent Application Publication
(10) Pub. No.: US 2026/0354769 A1
(43) Pub. Date: Nov. 12, 2026

(54) EVALUATING EFFECTIVE AGE WITH PHYSIOLOGICAL MONITORING

(71) Applicant: Whoop, Inc., Boston, MA (US)

(72) Inventors: Victoria Harrison Lee, Waltham, MA (US); Laura Ware, Boston, MA (US); Christopher Bailey, Cambridge, MA (US); Emily Smith, Boston, MA (US); Dylan Baroody, Boston, MA (US); Martin Oberhäuser, Hamburg (DE); Emily Rachel Capodilupo, Boston, MA (US); Simon Meuten, Hamburg (DE)

(21) Appl. No.: 19/461,616

(22) Filed: Jan. 27, 2026

Related U.S. Application Data
(60) Provisional application No. 63/750,181, filed on Jan. 27, 2025.

(51) Int. Cl. A61B 5/00; A61B 5/024; A61B 5/083; A61B 5/11

(52) U.S. Cl. CPC A61B 5/7275; A61B 5/0002; A61B 5/02405; A61B 5/0833; A61B 5/112; A61B 5/4815; A61B 5/4869; A61B 5/742

(57) ABSTRACT

An effective age for a user is calculated based on a comparison of metrics derived from continuous physiological monitoring data to all-cause mortality data. The effective age may synthesize multiple inferences available from different health metrics in a manner that couples the underlying physiological data to correlations identified in published literature, while avoiding double-counting of the contributions of underlying physiological data to an aggregated effective age. Furthermore, the effective age may be presented to a user in a manner that decomposes the effective age into different age adjustments associated with different metrics in order to surface actionable user insights for mitigating health risks associated with aging. This approach advantageously presents the results of continuous monitoring to a user in a manner that encourages concrete and measurable behavioral interventions for long-term health.

### RELATED APPLICATIONS

This application claims priority to U.S. Prov. App. No. 63/750,181 filed on Jan. 27, 2025, the entire content of which is hereby incorporated by reference.

This application is also related to International Patent App. No. PCT/US26/12771 filed on Jan. 27, 2026, which claims priority to U.S. Prov. App. No. 63/750,181 filed on Jan. 27, 2025, where the entire content of each of the foregoing is hereby incorporated by reference.

### BACKGROUND

A wearable fitness monitor can generate a variety of metrics. However, it can be difficult or impossible for a user to integrate multiple data sources and arrive at suitable behavioral interventions for long-term health. There remains a need for improved reporting of data-driven health states and recommended behavioral interventions.

### SUMMARY

An effective age for a user is calculated based on a comparison of metrics derived from continuous physiological monitoring data to all-cause mortality data. The effective age may synthesize multiple inferences available from different health metrics in a manner that couples the underlying physiological data to correlations identified in published literature, while avoiding double counting of the contributions of underlying physiological data to an aggregated effective age. Furthermore, the effective age may be presented to a user in a manner that decomposes the effective age into different age adjustments associated with different metrics in order to surface actionable user insights for mitigating health risks associated with aging. This approach advantageously presents the results of continuous monitoring to a user in a manner that encourages concrete and measurable behavioral interventions for long-term health.

A computer program product as described herein includes computer-executable code embodied in a non-transitory computer-readable medium that, when executing on one or more computing devices, causes the one or more computing devices to perform the steps of: acquiring data from a physiological monitor worn by a user, the data including cardiac activity data for the user and movement data for the user; calculating a plurality of metrics for the user based on the data from the physiological monitor, the plurality of metrics including at least: a resting heart rate for the user based on the cardiac activity data; an exercise metric for the user based on the cardiac activity data recorded during an exercise activity, a sleep metric derived from the data for the user, and a movement metric for the user derived from the movement data for the user; calculating a first plurality of age adjustments based on the plurality of metrics; acquiring a plurality of health data measurements for the user including at least: a chronological age of the user, a body mass index of the user, and a maximal oxygen consumption for the user; calculating a second plurality of age adjustments based on the plurality of health data measurements; and calculating an effective age of the user by adjusting the chronological age of the user according to each of the first plurality of age adjustments and each of the second plurality of age adjustments.

The computer program product may include computer-executable code that causes the one or more computing devices to perform the step of displaying the effective age of the user on a user device. The computer program product may include computer-executable code that causes the one or more computing devices to perform the step of displaying the effective age, the chronological age, and a pace of aging of the user based on a difference between the chronological age and the effective age. The maximal oxygen consumption for the user may be estimated based on the data from the physiological monitor. The resting heart rate may be measured during an automatically detected sleep interval for the user. The sleep metric may include one or more of a quantity of sleep, a sleep debt, and a sleep consistency. The plurality of metrics may include a movement metric based on detecting steps by the user with the movement data. The movement metric may be based on one or more of strength training repetitions, a daily step count for the user, a timing of sedentary and active intervals, a distance traveled during physical activity by the user, and an activity categorization based on the movement data. The exercise metric may include an average daily time within one or more target heart rate zones. One or more of the plurality of metrics may be averaged over at least seven days.

In another aspect, a method disclosed herein includes acquiring data for a user from a physiological monitor worn by the user; calculating a plurality of health metrics for the user from the data; calculating a plurality of age adjustments for the user based on the plurality of health metrics, wherein each of the plurality of age adjustments may be derived from a hazard ratio for a corresponding health metric based on an all-cause mortality rate for a population of users; calculating a total age adjustment for the user based on a weighted sum of the plurality of age adjustments; and calculating an effective age for the user by adjusting a chronological age of the user according to the total age adjustment.

The method may include displaying the effective age to the user. The data for the user may include at least one of cardiac data and movement data. The plurality of health metrics may include one or more of a resting heart rate for the user, a heart rate variability for the user, a daily step count for the user, a strength metric, and a maximal oxygen consumption for the user. The plurality of health metrics may include a sleep metric measuring at least one of a quantity of sleep, a quality of sleep, a quantity of restorative sleep, and a consistency of sleep. The plurality of health metrics may include a strain score based on heart rate zones for one or more exercise activities by the user. A common set of the data from the physiological monitor may be used to derive two or more of the plurality of health metrics, and a plurality of weights for the weighted sum of the plurality of age adjustments may be statistically derived to mitigate double counting of an age impact of the data among the plurality of age adjustments derived for the plurality of health metrics.

In another aspect, a system disclosed herein includes a physiological monitor configured to acquire data for a user; one or more processors configured by computer-executable code stored in a memory to perform the steps of: receiving the data from the physiological monitor, calculating a plurality of health metrics for the user from the data, calculating a plurality of age adjustments for the user based on the plurality of health metrics, wherein each of the plurality of age adjustments may be derived from a hazard ratio for a corresponding health metric based on an all-cause mortality rate for a population of users, calculating a total age adjustment for the user based on a weighted sum of the plurality of age adjustments, and calculating an effective age for the user by adjusting a chronological age of the user according to the total age adjustment; and a user device configured to display the effective age to the user.

The one or more processors may include processing resources on the user device. The one or more processors may include processing resources of a remote server coupled in a communicating relationship with the physiological monitor and the user device.

In another aspect, a computer program product disclosed herein includes computer-executable code embodied in a non-transitory computer-readable medium that, when executing on one or more computing devices, causes the one or more computing devices to perform the steps of: acquiring data for a user from a physiological monitor worn by the user; calculating a plurality of health metrics for the user from the data; calculating a plurality of age adjustments for the user based on the plurality of health metrics, wherein each of the plurality of age adjustments may be derived from a hazard ratio for a corresponding health metric based on an all-cause mortality rate for a population of users; calculating a total age adjustment for the user based on a weighted sum of the plurality of age adjustments; calculating an effective age for the user by adjusting a chronological age of the user according to the total age adjustment; displaying age-related data to the user including a pace of aging of the user relative to a pace of chronological aging of the user; and providing coaching to the user to improve the pace of aging based on one of the plurality of health metrics.

Displaying age-related data may include displaying the chronological age of the user and the effective age of the user. Displaying age-related data may include displaying each of the plurality of age adjustments and the total age adjustment applied to the chronological age. Displaying age-related data may include displaying a difference between the effective age and the chronological age. Providing coaching may include providing a fitness goal that impacts the effective age and that can be monitored for progress by the physiological monitor. The fitness goal may include a goal for one of the plurality of health metrics. The fitness goal may include at least one of a resting heart rate, a maximal oxygen consumption, and a body mass index. The the fitness goal may include a behavior used for calculating the effective age. The fitness goal may include one or more of a daily step count, a strength training goal, a weekly heart rate zone time, and a sleep goal. The sleep goal may include a measure of at least one of quantity of sleep, sleep debt, or sleep consistency. Providing coaching may include displaying a plurality of fitness goals to the user in an interface configured to receive a selection of a selected fitness goal from the plurality of fitness goals and, in response to the selection, to generate a plan for the user to progress toward the selected fitness goal.

In another aspect, a method disclosed herein includes acquiring data for a user from a physiological monitor worn by the user; calculating a plurality of health metrics for the user from the data; calculating a plurality of age adjustments for the user based on the plurality of health metrics, wherein each of the plurality of age adjustments may be derived from a hazard ratio for a corresponding health metric based on an all-cause mortality rate for a population of users; calculating a total age adjustment for the user based on a weighted sum of the plurality of age adjustments; calculating an effective age for the user by adjusting a chronological age of the user according to the total age adjustment; and displaying age-related data to the user based on a pace of aging of the user relative to a pace of chronological aging.

Displaying age-related data may include displaying the chronological age, the effective age, and the pace of aging of the user. Displaying age-related data may include displaying a health score based on the effective age of the user. The method may include providing coaching for the user to reduce the effective age. The method may include monitoring user activity relative to one or more coaching recommendations and providing feedback to the user concerning progress toward one or more fitness goals associated with the effective age. Displaying age-related data may include displaying a graphic to the user that encodes information about the effective age. The graphic may include a color based on a magnitude of a difference between the effective age and the chronological age. The graphic may include a perturbed circle with a magnitude and a number of deviations in a perimeter of the perturbed circle increasing inversely in proportion to the pace of aging. The graphic may include an animation that varies in speed according to an age-related metric for the user.

In another aspect, a system described herein includes a physiological monitor including one or more sensors, the physiological monitor configured to acquire data for a user with the one or more sensors and transmit the data to a remote resource; a server configured to receive the data from the physiological monitor and calculate an effective age for the user as a weighted sum of a plurality of age adjustments, wherein: each of the plurality of age adjustments may be derived from a hazard ratio for one or more health metrics based on an all-cause mortality rate associated with the one or more health metrics, each of the one or more health metrics may be derived from the data for the user received from the physiological monitor, and a plurality of weights for the weighted sum are statistically derived to avoid double counting of a contribution of each of the one or more health metrics to the effective age of the user; and a user device coupled to the server, the user device configured to receive age-related data based on the effective age from the server and display the age-related data to the user.

The data may include motion data from the physiological monitor. The data may include cardiac data from the physiological monitor. The data may include geolocation data from the physiological monitor. The physiological monitor may include an item of exercise equipment, further wherein the data may include activity data from the item of exercise equipment during operation by the user. The physiological monitor may include an electronic scale, further wherein the data may include weight measurements obtained from the electronic scale while weighing the user.

### BRIEF DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, with emphasis instead being placed upon illustrating the principles of the devices, systems, and methods described herein. In the drawings, like reference numerals generally identify corresponding elements.

FIG. 1 shows a physiological monitoring device.

FIG. 2 shows a physiological monitoring system.

FIG. 3 shows a sensing system.

FIG. 4A shows examples of physiological monitoring devices.

FIG. 4B shows examples of physiological monitoring devices.

FIG. 4C shows examples of physiological monitoring devices.

FIG. 5 shows a smart garment system.

FIG. 6 shows a method for evaluating an effective age of a user based on physiological data.

FIG. 7 illustrates a user interface for tracking effective age and other age-related data.

FIG. 8 shows health recommendations for a number of health metrics.

FIG. 9 illustrates an effective age calculation.

FIG. 10 illustrates age adjustments for sleep consistency.

FIG. 11 illustrates age adjustments for strength training.

FIG. 12 illustrates age adjustments for lean body mass (percentage).

FIG. 13 illustrates age adjustments for VO₂ max.

FIG. 14 illustrates age adjustments for sleep duration.

### DESCRIPTION

The embodiments will now be described more fully hereinafter with reference to the accompanying figures in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “user” as used herein, refers to any type of animal, human or non-human, whose physiological information may be monitored using an exemplary wearable physiological monitoring device and/or system.

The term “continuous,” as used herein in connection with heart rate data, refers to the acquisition of heart rate data at a sufficient frequency to enable detection of individual heartbeats, and also refers to the collection of heart rate data over extended periods, such as an hour, a day or more (including acquisition throughout the day and night), etc. More generally, with respect to physiological signals that might be monitored by a wearable device, “continuous” or “continuously” will be understood to mean continuously at a rate and duration suitable for the intended time-based processing, and physically at an inter-periodic rate (e.g., multiple times per heartbeat, respiration, and so forth) sufficient for resolving the desired physiological characteristics, such as heart rate, heart rate variability, heart rate peak detection, pulse shape, and so forth. Continuous monitoring should also be understood to include periodic sampling at any suitable interval, duration, and frequency. Thus, for example, continuous monitoring may include measuring a user's body temperature once every ten minutes, or monitoring heart activity by alternately sampling the heart rate for a minute and then pausing sampling for a minute, e.g., to conserve power or memory at times when the measured heart rate indicates that the user is at rest. Sampling may also be dynamic based on sensor input, for example, increasing the sampling rate when signal variability increases, or during periods of relatively higher motion, or based on user input.

At the same time, continuous monitoring is not intended to exclude ordinary data acquisition interruptions, such as temporary displacement of monitoring hardware due to sudden movements, changes in external lighting, loss of electrical power, physical manipulation and/or adjustment by a wearer, physical displacement of monitoring hardware due to external forces, and so forth. It will also be noted that heart rate data or a monitored heart rate, in this context, may more generally refer to raw sensor data, such as optical intensity signals, or processed data therefrom, such as heart rate data, signal peak data, heart rate variability data, or any other physiological or digital signal suitable for recovering heart rate information as contemplated herein. Furthermore, such heart rate data may generally be captured over some historical period that can be subsequently correlated to various other data or metrics related to, e.g., sleep states, recognized exercise activities, resting heart rate, maximum heart rate, and so forth.

The term “computer-readable medium,” as used herein, refers to a non-transitory storage media such as storage hardware, storage devices, computer memory that may be accessed by a controller, a microcontroller, a microprocessor, a computational system, or the like, or any other module or component of a computational system to encode thereon computer-executable instructions, software programs, and/or other data. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), virtual or physical computer system memory, physical memory hardware such as random access memory (such as DRAM, SRAM, EDO RAM), and so forth. Although not depicted, any of the devices or components described herein may include a computer-readable medium or other memory for storing program instructions, data, and the like.

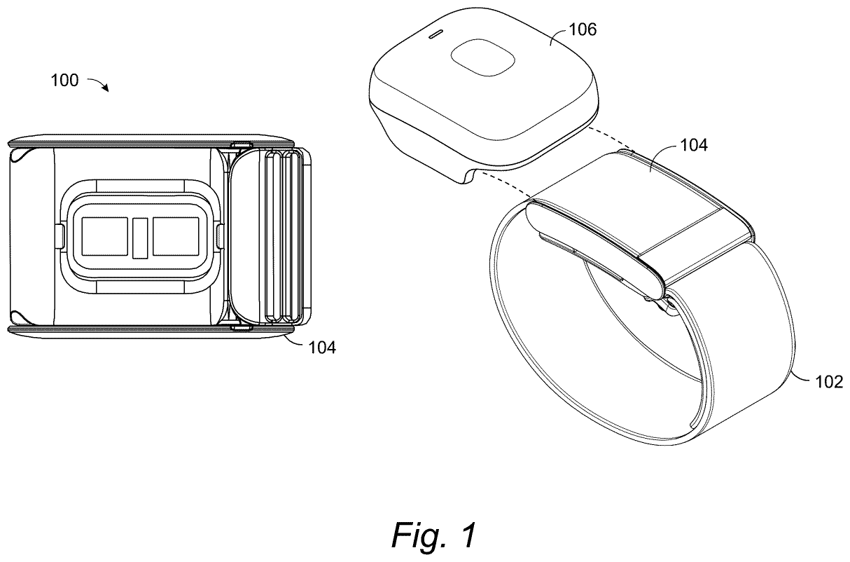

FIG. 1 shows a physiological monitoring system. The system 100 may include a wearable monitor 104 that is configured for physiological monitoring. The system 100 may also include a removable and replaceable battery 106 for recharging the wearable monitor 104. The wearable monitor 104 may include a strap 102 or other retaining system(s) for securing the wearable monitor 104 in a position on a wearer's body for the acquisition of physiological data as described herein. For example, the strap 102 may include a slim elastic band formed of any suitable elastic material such as rubber or a woven polymer fiber such as woven polyester, polypropylene, nylon, spandex, and so forth. The strap 102 may be adjustable to accommodate different wrist sizes, and may include any latches, hasps, or the like to secure the wearable monitor 104 in an intended position for monitoring a physiological signal. While a wrist-worn device is depicted, it will be understood that the wearable monitor 104 may be configured for positioning in any suitable location on a user's body, based on the sensing modality and the nature of the signal to be acquired. For example, the wearable monitor 104 may be configured for use on a wrist, a forearm, an ankle, a lower leg, a bicep, a chest, side torso, back, a gluteus, behind the ear, forehead, or any other suitable location(s), and the strap 102 may be, or may include, a waistband or other elastic band or the like within an article of clothing or accessory. In another aspect, the wearable monitor 104 may be configured as a ring, earring, stick-on, clip-on, head-mounted (e.g., glasses or goggles), or other article of clothing or accessory that can be worn by a user, and that contains suitable instrumentation, memory, and/or processing for physiological monitoring as described herein. The wearable monitor 104 may also or instead be structurally configured for placement on or within a garment, e.g., permanently or in a removable and replaceable manner. To that end, the wearable monitor 104 may be shaped and sized for placement within a pocket, slot, and/or other housing that is coupled to or embedded within a garment. In such configurations, the pocket or other retaining arrangement on the garment may include sensing windows or the like so that the wearable monitor 104 can operate while placed for use in the garment. U.S. Pat. No. 11,185,292 and U.S. Pat. Pub. No. 2024/0106283 describe non-limiting example embodiments of suitable wearable monitors 104 and are incorporated herein by reference in their entirety. And while the present disclosure may refer to a wrist-worn wearable or other wearable, it should be understood that any of the other locations or forms described herein are also included unless expressly stated to the contrary or otherwise clear from the context.

The system 100 may include any hardware components, subsystems, and the like to support various functions of the wearable monitor 104, such as data collection, processing, display, and communication with external resources. For example, the system 100 may include hardware for a heart rate monitor using, e.g., photoplethysmography, electrocardiography, or any other technique(s). The system 100 may be configured such that, when the wearable monitor 104 is placed for use about a wrist (or at some other body location), the system 100 initiates acquisition of physiological data from the wearer. In some embodiments, the pulse or heart rate may be acquired optically based on a light source (such as light-emitting diodes (LEDs)) and optical detectors in the wearable monitor 104. The LEDs may be positioned to direct illumination toward the user's skin, and optical detectors such as photodiodes may be used to capture illumination intensity measurements indicative of illumination from the LEDs that is reflected and/or transmitted by or through the wearer's skin, or depending on the configuration, through capillaries or arteries.

The system 100 may be configured to record other physiological and/or biomechanical parameters including, but not limited to, skin temperature (using a thermometer), galvanic skin response (using a galvanic skin response sensor), motion (using one or more multi-axis accelerometers and/or a gyroscope), blood pressure (via physical pressure measurements or other means), sound, electrocardiograms, and the like, as well as environmental or contextual parameters such as ambient light, ambient temperature, humidity, time of day, location, and so forth. For example, the wearable monitor 104 may include sensors such as accelerometers and/or gyroscopes for motion detection, sensors for environmental temperature sensing, sensors to measure electrodermal activity (EDA), sensors to measure galvanic skin response (GSR) sensing, and so forth. The system 100 may also or instead include other systems or subsystems supporting additional functions of the wearable monitor 104. For example, the system 100 may include communication systems to support, e.g., near-field communications, proximity sensing, touch sensing (e.g., via capacitive or resistive sensors), Bluetooth communications, Wi-Fi communications, cellular communications, satellite communications, and so forth. The wearable monitor 104 may also or instead include components such as a GeoPositioning System (GPS), a display and/or user interface, a clock and/or timer, and so forth.

The wearable monitor 104 may include one or more sources of battery power, such as a first battery within the wearable monitor 104 and a second battery 106 that is removable from and replaceable to the wearable monitor 104 in order to recharge the battery in the wearable monitor 104. The wearable monitor 104 may also or instead include systems for energy harvesting via, e.g., kinetic energy capture, ambient electromagnetic radiation capture, solar/optical energy capture, and so forth, as well as systems for short and/or medium-range wireless energy transfer to receive power from nearby wireless power sources. Also or instead, the system 100 may include a plurality of wearable monitors 104 (and/or other physiological monitors) that can share battery power or provide power to one another, e.g., using a garment power infrastructure, wireless power sharing network, or the like. The system 100 may perform numerous functions related to continuous monitoring, such as automatically detecting when the user is asleep, awake, exercising, and so forth, and such detections may be performed locally at the wearable monitor 104 or at a remote service such as a mobile device or cloud computing resource coupled in a communicating relationship with the wearable monitor 104 and receiving data therefrom. In general, the system 100 may support continuous, independent monitoring of a physiological signal such as a heart rate, and the underlying acquired data may be stored on the wearable monitor 104 for an extended period until it can be uploaded to a remote processing resource for more computationally complex analysis. In one aspect, the wearable monitor 104 may be a wrist-worn photoplethysmography device, although other form factors are also or instead possible as described herein, such as a ring, a bicep band, a calf band, an elastic band in a garment, a patch, a clip-on device, and so forth.

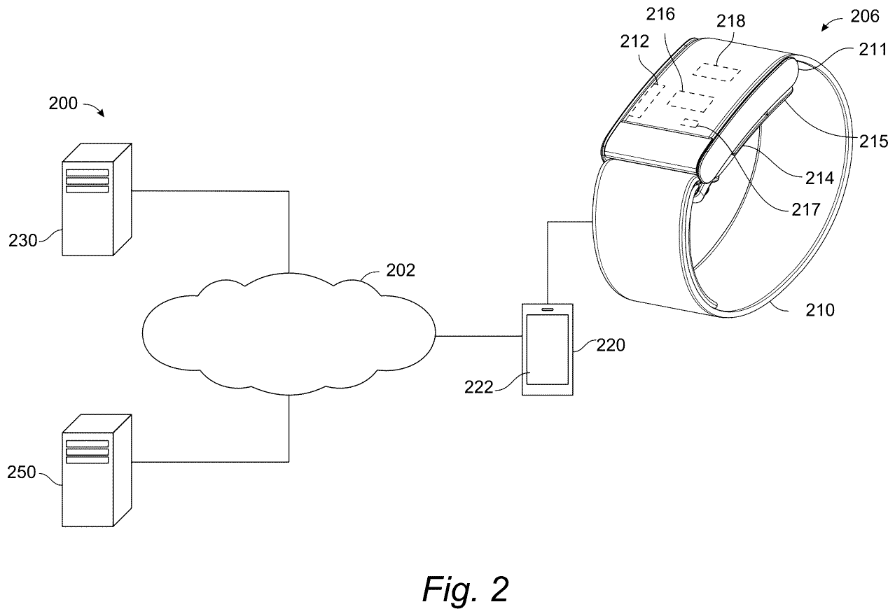

FIG. 2 illustrates a physiological monitoring system. More specifically, FIG. 2 illustrates a physiological monitoring system 200 that may be used with any of the methods or devices described herein. In general, the system 200 may include a physiological monitor 206, a user device 220, a remote server 230 with a remote data processing resource (such as any of the processors or processing resources described herein), and one or more other resources 250, all of which may be interconnected through a data network 202.

The data network 202 may be any of the data networks described herein. For example, the data network 202 may be any network(s) or internetwork(s) suitable for communicating data and information among participants in the system 200. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 802.16m)), fifth generation (e.g., 5G), and/or other technologies, as well as any of a variety of corporate area or local area networks and other switches, routers, hubs, gateways, and the like that might be used to carry data among participants in the system 200. This may also include local or short-range communications infrastructure suitable, e.g., for coupling the physiological monitor 206 to the user device 220 or otherwise supporting communication with local resources. By way of non-limiting examples, short-range communications may include Wi-Fi communications, Bluetooth communications, infrared communications, near-field communications, communications with RFID tags or readers, and so forth.

The physiological monitor 206 may, in general, be any physiological monitoring device or system, such as any of the wearable monitors or other monitoring devices or systems described herein. In one aspect, the physiological monitor 206 may be a wearable physiological monitor shaped and sized to be worn on a wrist or other body location. The physiological monitor 206 may include a wearable housing 211, a network interface 212, one or more sensors 214, one or more light sources 215, a processor 216, a haptic device 217 or other user input/output hardware, a memory 218, and a strap 210 for retaining the physiological monitor 206 in a desired location on a user. In one aspect, the physiological monitor 206 may be configured to acquire heart rate data and/or other physiological data from a wearer in an intermittent or substantially continuous manner. In another aspect, the physiological monitor 206 may be configured to support extended, continuous acquisition of physiological data, e.g., for several days, a week, or more.

The network interface 212 of the physiological monitor 206 may be configured to couple the physiological monitor 206 to one or more other components of the system 200 in a communicating relationship, either directly, e.g., through a cellular data connection or the like, or indirectly through a short-range wireless communications channel coupling the physiological monitor 206 locally to a wireless access point, router, computer, laptop, tablet, cellular phone, or other device that can locally process data, and/or relay data from the physiological monitor 206 to the remote server 230 or other resource(s) 250 as necessary or helpful for acquiring and processing data from the physiological monitor 206. The network interface 212 may also or instead facilitate connections among multiple wearable devices, power sources, and the like, e.g., in a wearable device area network or other multi-device monitoring infrastructure.

The one or more sensors 214 may include any of the sensors described herein, or any other sensors or sub-systems suitable for physiological monitoring or supporting functions. By way of example and not limitation, the one or more sensors 214 may include one or more of a light source (including, e.g., LEDs or other wavelength-specific sources of green light, red light, infrared light, and so forth, as well as broadband illumination), an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, and so forth. The one or more sensors 214 may also or instead include sensors (and accompanying hardware/software) for, e.g., a Global Positioning System, a proximity sensor, an RFID tag reader, an RFID tag, a temporal sensor, an electrodermal activity sensor, an electrocardiogram, a pressure sensor, an acoustic sensor (e.g., a microphone), a camera (e.g., visible light and/or infrared), and the like. The one or more sensors 214 may be disposed in the wearable housing 211 or otherwise positioned and configured for physiological monitoring or other functions described herein. In one aspect, the one or more sensors 214 include a light detector configured to provide light intensity data to the processor 216 (or to the remote server 230) for calculating a heart rate and a heart rate variability. The one or more sensors 214 may also or instead include an accelerometer, gyroscope, and the like configured to provide motion data to the processor 216, e.g., for detecting activities such as a sleep state, a resting state, a waking event, exercise, and/or other user activity. In an implementation, the one or more sensors 214 may include a sensor to measure a galvanic skin response of the user. The one or more sensors 214 may also or instead include electrodes or the like for capturing electronic signals, e.g., to obtain an electrocardiogram and/or other electrically-derived physiological measurements.

The processor 216 and memory 218 may be any of the processors and memories described herein. In one aspect, the memory 218 may store physiological data obtained by monitoring a user with the one or more sensors 214, and/or any other sensor data, program data, or other data useful for operation of the physiological monitor 206 or other components of the system 200. It will be understood that, while only the memory 218 on the physiological monitor is illustrated, any other device(s) or components of the system 200 may also or instead include a memory to store program instructions, raw data, processed data, user inputs, and so forth. In one aspect, the processor 216 of the physiological monitor 206 may be configured to obtain heart rate data from the user, such as heart rate data including or based on the raw data from the sensors 214. The processor 216 may also or instead be configured to determine, or assist in a determination of, a condition of the user related to, e.g., health, fitness, strain, recovery, sleep, or any of the other conditions described herein.

The one or more light sources 215 may be coupled to the wearable housing 211 and controlled by the processor 216. At least one of the light sources 215 may be directed toward the skin of a user adjacent to the wearable housing 211. Light from the light source 215, or more generally, light at one or more wavelengths of the light source 215, may be detected by one or more of the sensors 214, and processed by the processor 216 as described herein.

The system 200 may further include a remote data processing resource executing on a remote server 230. The remote data processing resource may include any of the processors and related hardware described herein, and may be configured to receive data transmitted from the memory 218 of the physiological monitor 206, and to process the data to detect or infer physiological signals of interest such as heart rate, heart rate variability, respiratory rate, pulse oxygen, blood pressure, and so forth. The remote server 230 may also or instead evaluate a condition of the user such as a recovery state, sleep state, exercise activity, exercise type, sleep quality, daily activity strain, and any other health or fitness conditions that might be detected based on such data.

The system 200 may include one or more user devices 220, which may work together with the physiological monitor 206, e.g., to provide a display, or more generally, user input/output, for user data and analysis, and/or to provide a communications bridge from the network interface 212 of the physiological monitor 206 to the data network 202 and the remote server 230. For example, the physiological monitor 206 may communicate locally with a user device 220, such as a smartphone of a user, via short-range communications, e.g., Bluetooth, or the like, for the exchange of data between the physiological monitor 206 and the user device 220, and the user device 220 may in turn communicate with the remote server 230 via the data network 202 in order to forward data from the physiological monitor 206 and to receive analysis and results from the remote server 230 for presentation to the user. In one aspect, the user device(s) 220 may support physiological monitoring by processing or pre-processing data from the physiological monitor 206 to support extraction of heart rate or heart rate variability data from raw data obtained by the physiological monitor 206. In another aspect, computationally intensive processing may advantageously be performed at the remote server 230, which may have greater memory capabilities and processing power than the physiological monitor 206 and/or the user device 220.

The user device 220 may include any suitable computing device(s) including, without limitation, a smartphone, a desktop computer, a laptop computer, a network computer, a tablet, a mobile device, a portable digital assistant, a cellular phone, a portable media or entertainment device, or any other computing devices described herein, including, e.g., supplemental wearable devices and/or computers. The user device 220 may provide a user interface 222 for access to data and analysis by a user, and/or to support user control of operation of the physiological monitor 206. The user interface 222 may be maintained by one or more applications executing locally on the user device 220, or the user interface 222 may be remotely served and presented on the user device 220, e.g., from the remote server 230 or the one or more other resources 250.

In general, the remote server 230 may include data storage, a network interface, and/or other processing circuitry. The remote server 230 may process data from the physiological monitor 206 and perform physiological and/or health monitoring/analyses or any of the other analyses described herein (e.g., analyzing sleep, determining strain, assessing recovery, and so on), and may host a user interface for remote access to this data, e.g., from the user device 220. The remote server 230 may include a web server or other programmatic front end that facilitates web-based access by the user devices 220 or the physiological monitor 206 to the capabilities of the remote server 230 or other components of the system 200.

The system 200 may include other resources 250, such as any resources that can be usefully employed in the devices, systems, and methods as described herein. For example, these other resources 250 may include other data networks, databases, processing resources, cloud data storage, data mining tools, computational tools, data monitoring tools, algorithms, and so forth. In another aspect, the other resources 250 may include one or more administrative or programmatic interfaces for human actors, such as programmers, researchers, annotators, editors, analysts, coaches, and so forth, to interact with any of the foregoing. The other resources 250 may also or instead include any other software or hardware resources that may be usefully employed in the networked applications as contemplated herein. For example, the other resources 250 may include payment processing servers or platforms used to authorize payment for access, content, or option/feature purchases. In another aspect, the other resources 250 may include certificate servers or other security resources for third-party verification of identity, encryption or decryption of data, and so forth. In another aspect, the other resources 250 may include a desktop computer or the like co-located (e.g., on the same local area network with, or directly coupled to through a serial or USB cable) with a user device 220, wearable strap 210, or remote server 230. In this case, the other resources 250 may provide supplemental functions for components of the system 200, such as firmware upgrades, user interfaces, and storage and/or pre-processing of data from the physiological monitor 206 before transmission to the remote server 230.

The other resources 250 may also or instead include one or more web servers that provide web-based access to and from any of the other participants in the system 200. While depicted as a separate network entity, it will be readily appreciated that the other resources 250 (e.g., a web server) may also or instead be logically and/or physically associated with one of the other devices described herein, and may, for example, include or provide a user interface 222 for web access to the remote server 230 or a database or other resource(s) to facilitate user interaction through the data network 202, e.g., from the physiological monitor 206 or the user device 220.

In another aspect, the other resources 250 may include fitness equipment or other fitness infrastructure. For example, a strength training machine may automatically record repetitions and/or added weight during repetitions, which may be wirelessly accessible by the physiological monitor 206 or some other user device 220. More generally, a gym may be configured to track user movement from machine to machine, and report activity from each machine in order to track various strength training activities in a workout. The other resources 250 may also or instead include other monitoring equipment or infrastructure. For example, the system 200 may include one or more cameras to track motion of free weights and/or the body position of the user during repetitions of a strength training activity or the like, and/or the cameras may be integrated into the physiological monitor 206 or other user device 220. Similarly, a user may wear, or have embedded in clothing, tracking fiducials such as visually distinguishable objects for image-based tracking, or radio beacons or the like for other tracking. In another aspect, weights may themselves be instrumented, e.g., with sensors to record and communicate detected motion, and/or beacons or the like to self-identify type, weight, and so forth, in order to facilitate automated detection and tracking of exercise activity with other connected devices.

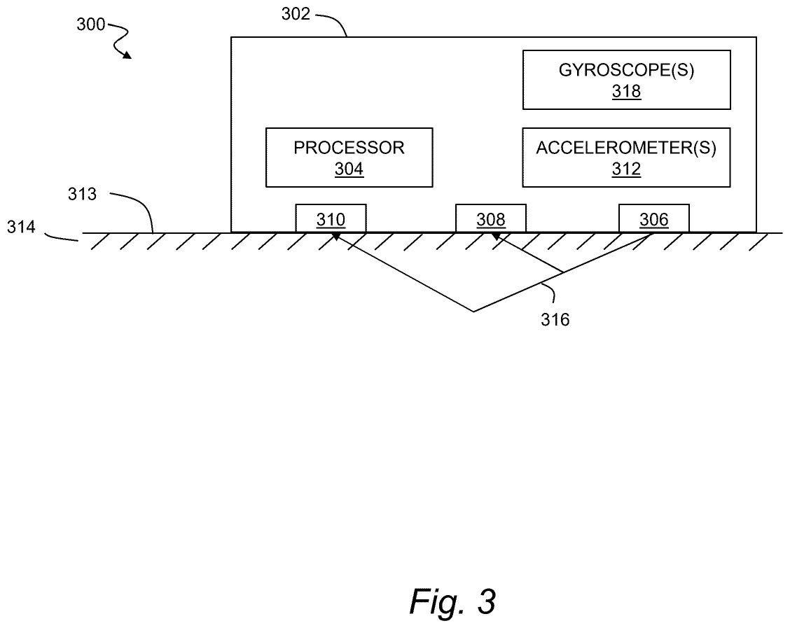

FIG. 3 shows a sensing system. In general, the system 300 may include a physiological monitor 302 with a processor 304, a light source 306, a first sensor 308 (e.g., a first photodetector), a second sensor 310 (e.g., a second photodetector), one or more accelerometers 312, one or more gyroscopes 318, and any other hardware or other components and systems suitable for physiological monitoring as described herein. The physiological monitor 302 may be positioned for use against a surface 313 of the skin 314 of a user where the light source 306 and sensors 308, 310 can contact the skin 314 for acquisition of physiological data. Although not depicted, it will be understood that the physiological monitor 302 may generally be retained in position using any of the straps, garments, patches, bands, clamps, clips, or the like described herein, and/or integrated into other wearable garments, accessories, and the like, such as audio earbuds, earrings, or similar, glasses and/or other eyewear, a ring, a headband, and so forth.

The processor 304 may be any microprocessor, microcontroller, application-specific integrated circuit, or other processing circuitry or combination of the foregoing suitable for controlling operation of the physiological monitor and acquiring physiological data.

The light source 306 may include one or more light-emitting diodes or other sources of illumination, and may be positioned within the physiological monitor 302 such that, when the physiological monitor 302 is placed for use on the skin 314, the light source 306 directs illumination toward the skin 314 and the illumination is reflected back toward the sensors 308, 310 as indicated by arrows 316 (or transmitted through the tissue to one or more opposing sensors), where the intensity can be measured. In one aspect, the light source 306 may include light-emitting diodes that emit light in the green, red, infrared, near-infrared, or other suitable wavelength ranges, which can provide desired light transmission through human skin, facilitating low-power transmission of measurable illumination to the sensors 308, 310, although other illumination sources and wavelengths may also or instead be used.

The sensors 308, 310 may be oriented to contact the skin 314 when the physiological monitor 302 is placed for use on this skin 314, and positioned so that the sensors 308, 310 can capture illumination reflected and/or transmitted by the skin from the light source 306. In general, the sensors 308, 310 may include photodiodes, photodetectors, or any other sensor(s) responsive to illumination from the light source 306. This may include broadband optical sensors, narrowband optical sensors, filtered sensors, or the like. In general, a first sensor 308 may be positioned closer to the light source 306 than a second sensor 310 to facilitate detection of differential intensity in the measured wavelength(s). For example, the first sensor 308 may be positioned 1-4 millimeters from the light source 306 and the second sensor 310 may be positioned 2-8 millimeters from the light source, or about twice as far as the first sensor 308 from the light source 306.

Other spacings may also or instead be used depending on, e.g., the intensity of the light source 306, the sensitivity of the sensors 308, 310, the contact force of the physiological monitor 302 on the skin 314, the degree of incursion of ambient light, the physiological measurements/properties of interest, and so forth. In one aspect, the sensors may be linearly arranged in a straight line away from the light source 306. While this provides consistency in comparative measurements, it is not strictly required, and the sensors 308, 310 may be displaced in any of a number of directions away from the light source 306 provided they both contact the skin 314 in a manner that permits capture of light through the skin 314 from the light source 306. In another aspect, the physiological monitor 302 may include one or more other light sources and/or light sensors, which may be arranged to improve accuracy and/or provide redundancy for the contact detection, or to support other measurements such as oxygenation or skin thickness. This may include light sources/sensors using different ranges of wavelengths, different patterns of illumination, and so forth. In another aspect, the two sensors 308, 310 may be positioned at different distances from a perimeter of the physiological monitor 302 so that the sensors 308, 310 can acquire differential intensity values for ambient light incident on the skin and transmitted through the skin to the sensors 308, 310.

In operation, the processor 304 may acquire raw intensity data from the sensors 308, 310, and perform local calculations such as pre-processing raw data for heart rate measurements or evaluating whether the physiological monitor 302 is properly placed for use on the skin 314.

The accelerometer 312 may include, e.g., one or more single-axis or multi-axis accelerometers, which may usefully measure motion of the physiological monitor 302 to support calculations such as automated activity detection, device on/off evaluation, degree of musculoskeletal activation, and so forth. Other motion and orientation sensing hardware—such as one or more gyroscopes 318, inertial motion sensors, and/or other micro-electromechanical system (MEMS) sensors—may also or instead be used for these purposes. More generally, the physiological monitor 302 may include any additional components, subsystems, and the like suitable for supporting various modes of physiological monitoring and contextual data acquisition as described herein.

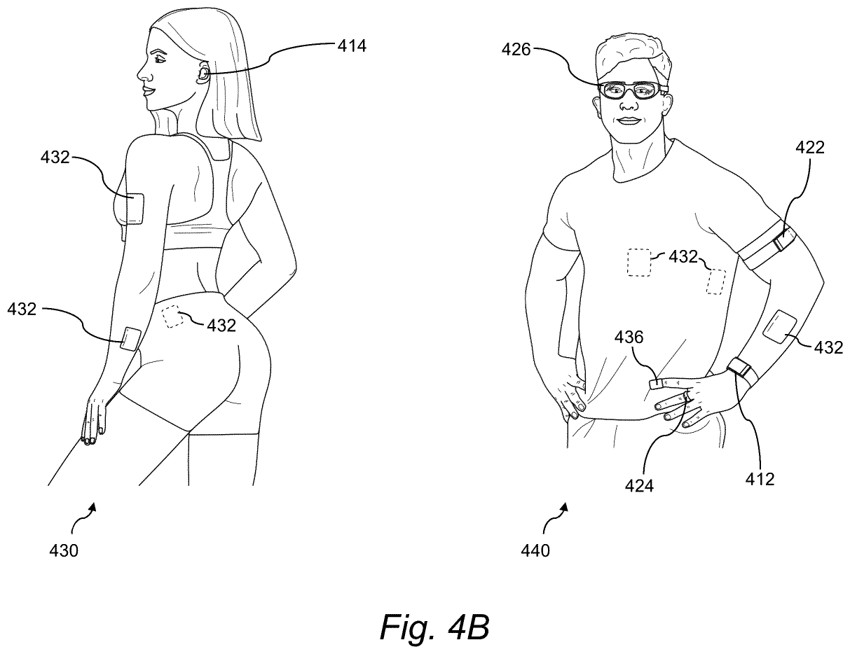

The physiological monitors described herein—e.g., in the systems 100, 200, 300 described above or elsewhere herein—may be provided in one or more different form factors. That is, although a wrist-worn device is illustrated in FIGS. 1 and 2, and garments with sensors are illustrated in FIG. 5, other form factors are also or instead possible, some of which are discussed below by way of example.

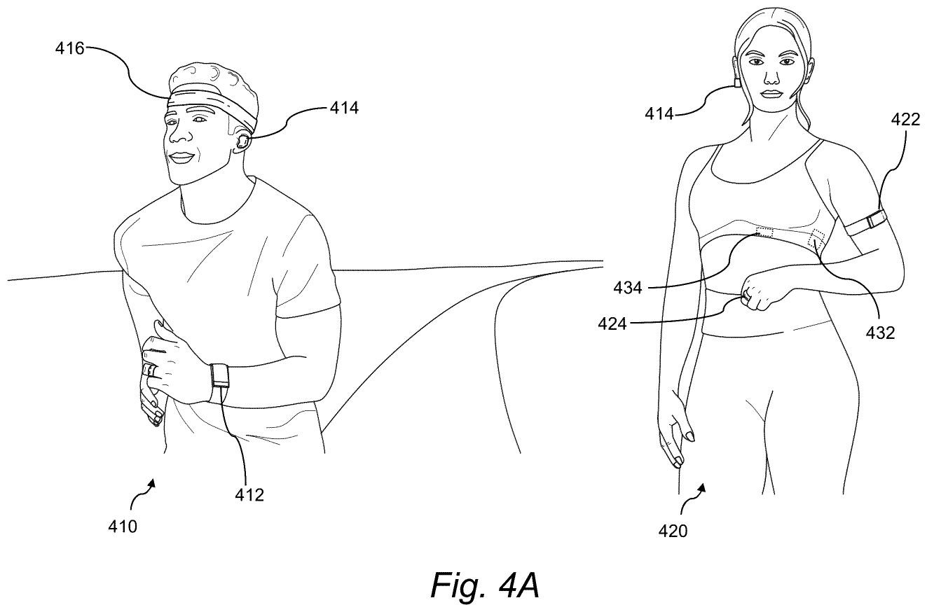

FIGS. 4A-4C illustrate physiological monitoring devices. The illustrated devices may include any of the hardware, software, and/or other components described herein for physiological sensing and/or other functions and may be embodied in various form factors for various use cases. These various form factors may be used individually or as multiple independent or cooperating physiological monitoring devices and may include two or more devices of the same type (e.g., two wrist-worn devices, two or more patches, and so on), and/or two or more different types of devices. Moreover, other form factors, and combinations thereof, may also or instead be used for physiological monitoring as described herein. It will further be understood that each of the different example form factors shown in these figures or elsewhere herein may include any one or more of the various sensors, emitters, processors, memories, interfaces, power supplies, and/or other processing and control circuitry, including without limitation any of the foregoing described herein, e.g., with reference to FIGS. 1-3 above.

FIG. 4A shows a first user 410 and a second user 420. The first user 410 may be wearing one or more physiological monitors, such as a wrist-worn device 412 (such as any described herein), an ear-worn device 414 (including on-ear devices retained with a clamp, clip, or other mechanism, and/or in-ear devices, such as earbuds or the like, that are retained at least in part within the ear canal), and a headband 416 or similar.

In one aspect, an ear-worn device 414 may be structurally configured to be partially or entirely inserted within an ear canal of the first user 410. In another aspect, the ear-worn device 414 may be configured to be worn on the ear lobe, or in some other location on the ear where, e.g., temperature, blood flow, respiration, and/or other physiological parameters can be measured. In one aspect, an ear-worn device 414 may be configured for heart rate monitoring, such as any of the heart rate monitoring described herein. For example, this may include continuous heart rate monitoring with optical sensors based on changes in blood volume beneath the skin. The ear-worn device 414 may also or instead be configured for temperature monitoring. For example, the ear-worn device 414 may include one or more infrared sensors, thermistors, thermocouples, or the like to measure the temperature of the ear canal and/or other surfaces. Surface measurements may also or instead be used to support other inferences about body temperature, heat dissipation, and the like, which may be related to current activity levels, general health and wellness, and so forth.

In another aspect, the ear-worn device 414, or any of the other devices described herein, may be configured for activity tracking. For example, the ear-worn device 414 may include one or more accelerometers, gyroscopes, Global Positioning System (GPS) sensors, and so forth to detect motion and provide information about physical activity levels. This may, for example, include large-scale motion, such as geographical movement and elevation changes, that can be tracked with GPS or the like, or local movement detected by the ear-worn device 414, which may be tracked with multi-axis gyroscopes, multi-axis accelerometers, and so forth. These latter sensors may be used to infer, e.g., steps taken, gait analysis, activity type, activity level, and/or overall movement.

The ear-worn device 414, or any of the other devices described herein, may also or instead be configured for blood pressure monitoring. This may, for example, include techniques based on cardiovascular waveform analysis (e.g., using the shape of a PPG or ECG signal from a single location), pulse transit time (e.g., based on the time difference between waveforms at two or more physical locations on the body with two or more monitors), pulse wave velocity (similar to pulse transit time, but over longer arterial distances), physical pulse monitoring (e.g., with pressure sensors, haptic stimulus responses, or other mechanical and/or dynamic techniques), tonometry (measuring the force required to counteract arterial pressure), oscillometric measurement (measuring oscillations in the arterial wall as a cuff deflates around a region of interest), volume clamping (measuring changes in pressure that are required to maintain constant blood volume in a region of interest), and so forth. Some of these blood pressure monitoring techniques are better suited to specific types and locations of monitors and may be more suited to, e.g., wristbands, bicep bands, chest straps, finger rings, and so forth, but are included here for completeness.

The ear-worn device 414, or any of the other devices described herein, may also or instead be configured for electrodermal activity (EDA) monitoring. For example, the ear-worn device 414 may include one or more electrodes in contact with the skin, which may be used to measure the electrical conductance thereof, and to infer, e.g., sweat levels, skin hydration, and/or other parameters correlated to skin conductance. Electrodes may also or instead be used for, e.g., ECG monitoring or the like.

The ear-worn device 414, or any of the other devices described herein, may also or instead be configured to sense blood oxygen saturation (also referred to as pulse oximetry or SpO₂) monitoring. To this end, the ear-worn device 414 may include one or more optical sources and detectors, and the system may use different absorption spectra of oxygenated and deoxygenated hemoglobin to estimate pulse oxygen saturation. In another aspect, the ear-worn device 414, or any of the other devices described herein, may be configured for brainwave monitoring, e.g., using electroencephalogram (EEG) sensors to monitor brainwave activity.

The ear-worn device 414, or any of the other devices described herein, may also or instead be configured for respiration rate monitoring. In one aspect, respiration rate may be inferred using respiratory sinus arrhythmia or other techniques to infer respiration rate from a measured heart rate signal over time. In another aspect, respiration rate may be inferred from physical changes in the ear canal (or chest, or other body part, where applicable to a particular sensor). Other techniques may also or instead be used. For example, the ear-worn device 414 may include a microphone or other audio transducer, and the respiration rate may be inferred from audio data acquired from the user.

In another aspect, a headband 416 may be structurally and programmatically configured for physiological sensing and/or monitoring using any of the systems and methods described herein. For example, the headband 416 may be configured to monitor heart rate, temperature, brain activity, electromyography, galvanic skin response, motion, activity, and so forth. In general, the sensors and processing may be adapted for the form factor of the headband 416. For example, the headband 416 may use temperature sensors to measure skin temperature and/or ambient temperature around the head. For brain activity, the headband 416 may include EEG sensors or the like embedded within the headband 416 to measure electrical activity in the brain, which can be used for monitoring brain waves associated with different states such as relaxation, concentration, and/or sleep. More generally, any physiological monitoring techniques described herein that can be adapted for use in a corresponding form factor may be deployed, either alone or in combination, for physiological monitoring with the headband 416. In another aspect, the headband 416 may incorporate a brain-computer interface (BCI) for control of a physiological monitoring system. This may, for example, include any system suitable for direct communication between the brain and external devices based on, e.g., signal acquisition using techniques such as electroencephalography, processing of these raw signals, feature extraction and translation, and then command execution based on an inferred user intention.

The second user 420 may be wearing one or more physiological monitors such as an ear-worn device 414 (which may be any as described herein, and which may be configured as a clamp, clip, earring, or similar, as shown), a bicep band 422, a ring 424, a patch 432 (such as any as described herein, e.g., with reference to FIG. 4B), and a band sensor 434.

The bicep band 422 may be configured for physiological monitoring and sensing using any of the systems and methods described herein, e.g., by retaining a sensor in place with the bicep band 422 or integrating components of the sensor into the bicep band 422, or some combination of these. The bicep band 422 may be configured to monitor heart rate, motion, activity, temperature, blood pressure, blood oxygen saturation, hydration, body composition, ultraviolet light exposure, electrodermal activity, and so forth, as well as combinations of the foregoing. In one aspect, electromyography (EMG) may be used to measure electrical activity in the muscles, e.g., with one or more electrical contacts or the like embedded in the bicep band 422, which can provide information about muscle contraction and fatigue during physical activity. Body composition analysis may be performed using, e.g., bioelectrical impedance analysis to estimate various components of body composition such as fat (percentage or mass), muscle (percentage or mass), and hydration. In another aspect, the bicep band 422 may include one or more sensors to measure ambient light, and more specifically, ambient ultraviolet (UV) light. This may be used to monitor UV exposure, and to provide recommendations to the user to meet certain healthy thresholds for, e.g., vitamin D synthesis, mood, and immune function, and/or to provide alerts concerning possible overexposure. In another aspect, the bicep band 422 or other form factors described herein may be adapted for gesture control based on the capture of motion signals and corresponding inferences of user intent. While a bicep band 422 is illustrated, it will be understood that similar bands for other body parts may also or instead be used, such as leg bands (or more specifically, thigh bands, calf bands, ankle bands, etc.), chest bands, abdomen bands, neck bands, wrist bands, and so forth.

The ring 424 may be configured for physiological monitoring and sensing using any of the systems and methods described herein. For example, the ring 424 may be configured to monitor heart rate, motion, activity, sleep, temperature, blood pressure, respiration rate, blood oxygen saturation, hydration, UV exposure, and so forth. A ring 424 is also advantageously positioned to capture a wide range of hand motions and may be configured for gesture control of physiological monitoring and/or related hardware and software. The ring 424 may be configured for wearing on a finger, as shown in the figure, or another portion of a wearer's body (e.g., a thumb, a toe, and so forth).

The band sensor 434 may be the same or similar to the other monitors described herein and/or any of the bands as described herein. In an aspect, the band sensor 434 may include a monitor inserted into (e.g., placed into a pocket or the like), coupled with, embedded within, or the like, a strap or band, e.g., an elastic band in an article of clothing, an accessory, or similar.

FIG. 4B shows a third user 430 and a fourth user 440. The third user 430 may be wearing one or more physiological monitors, such as an ear-worn device 414, which may be the same as or similar to any of those described herein, and one or more patches 432 that include sensors and the like to support physiological monitoring. By way of example, a patch 432 may be configured for physiological monitoring and sensing of heart rate monitoring, temperature, activity, motion, blood pressure, blood oxygen saturation, respiration rate, blood glucose, perspiration, hydration, ultraviolet exposure, and so forth, as well as combinations of the foregoing. In one aspect, the patch 432 may include a continuous glucose monitor with a sensor for insertion into fatty tissue under the skin, along with a transmitter to wirelessly transmit glucose data to a smartphone or other device. In another aspect, the patch 432 may include a hydration monitor using, e.g., electrical impedance analysis to measure resistance and reactance of body tissue with a small electrical current, or bioimpedance spectroscopy to measure impedance at various frequencies of electrical current. Hydration monitoring may also or instead use a wearable patch to collect sweat and analyze electrolyte concentrations correlated to hydration. Other techniques for measuring hydration using, e.g., near-infrared spectroscopy or capacitance hygrometry, may also or instead be employed where suitable adaptations can be made to any of the wearable monitors described herein. In another aspect, the patch 432, or any of the other monitors described herein, may be adapted to monitor environmental conditions, such as temperature, humidity, air quality, noise, light, and the like, that might be used to supplement physiological monitoring when evaluating the condition of a user. In another aspect, the patch 432, or any of the other monitors described herein, may be adapted for electrodermal activity monitoring, e.g., for tracking autonomic nervous system activity, stress, and the like based on galvanic skin response. One or more patches 432 may be coupled to a user in one or more of a plurality of locations on the body, such as those shown on the third user 430—e.g., a portion of an arm (e.g., the upper arm and/or the lower arm), and on or near the gluteus maximus, and similar. Other locations are also or instead possible, such as the chest, the abdomen, the forehead or temples, the wrist, a hand, a finger, a foot, a neck, a backside, the pelvic region, a portion of the back, a portion of a leg, and so forth.

The fourth user 440 may be wearing one or more physiological monitors, such as a bicep band 422, a wrist-worn device 412, a ring 424, and a patch 432, which may be the same or similar to any of the monitors described herein. The fourth user 440 further is shown with eyewear 426 and a fingertip monitor 436, as further explained below by way of example.

The eyewear 426 may include sensors or the like in contact areas or similar, such as a temple region, face region (e.g., via the frame or lens), or other head portion of the fourth user 440. For example, the eyewear 426 may be configured for physiological monitoring and sensing of heart rate, temperature, brain activity, motion, activity type, blood pressure, blood oxygen saturation, and so forth, as well as combinations of the foregoing. In one aspect, the eyewear 426 may employ electrooculography (EOG) to measure electrical activity of the muscles around the eyes or another region of the head/face, which can be used, e.g., to track eye movements and provide insights into cognitive states, attention levels, fatigue, and so forth. In another aspect, one or more EEG sensors may be integrated into the frame and/or temples of the eyewear 426 to measure electrical activity in the brain. The eyewear 426 may also or instead be configured to perform eye tracking using cameras and/or infrared or other sensors to monitor movement of the eyes, which can be used for various applications, including human-computer interaction, attention monitoring, and so forth. The eyewear 426 may also or instead be configured for augmented reality (AR) and virtual reality (VR) biometrics, e.g., where the eyewear 426 can include sensors that monitor physiological parameters to enhance user experience and safety, and to visually present information to the user related to any of the foregoing. In another aspect, the eyewear 426 may include cameras, microphones, and the like for recording and tracking environment information.

The fingertip monitor 436 may include a clamp, clip, or the like, and may be the same or similar to any of the physiological monitors described herein. In some aspects, the fingertip monitor 436 may include a pulse oximeter configured to measure oxygen saturation and/or heart rate for monitoring respiratory and/or cardiovascular health.

FIG. 4C shows the front and back of a fifth user 450 showing further example locations for a patch 432 or the like as described herein.

More generally, any one or more of the sensing modalities described herein may, provided suitable adaptations can be made, be deployed in any one or more of the wearable devices described herein. Furthermore, one or more of the wearable devices may communicate with one or more other wearable devices and/or with a control device such as a smartphone or other computing device, to perform cooperative monitoring. For example, various monitoring techniques, such as electrocardiography or blood pressure measurements using pulse transit time, may usefully be performed by combining signals from sensors at two or more different body locations, and a control device may usefully acquire signals from multiple devices and locations to perform such analysis. Similarly, multiple motion signals from different body locations may be used to refine activity detection, measure body temperature, and so forth. Thus, in one aspect, two or more wearable devices may cooperate with one another to perform an integrated sensing operation such as any of those described herein.

In another aspect, any one or more of the wearable electronic devices described herein may use energy harvesting to generate power from various external sources, and/or to supplement power supplied by an internal battery or the like. For example, a device may use solar energy harvesting to extract solar energy from ambient light sources. This may include integrating solar cells or other ambient light collectors into the wearable device to capture energy from sunlight and/or artificial light sources. In another aspect, the device may use kinetic energy harvesting to generate energy from movements by a user of the device. In another aspect, the device may use thermal energy harvesting to generate power based on differences between the body of the wearer and the surrounding environment. The device may also or instead use vibration energy harvesting, radio frequency energy harvesting (e.g., by capturing ambient RF signals, such as Wi-Fi or cellular signals, and converting them into usable electrical power), ambient light harvesting, and so forth. Other techniques may also or instead be used to provide external power, such as beam steering or resonant techniques for short-range or medium-range radio frequency power transfers. More generally, any technique or combination of techniques for powering a device, and/or for supplementing an internal power source such as a battery, with power from ambient sources may be used to power one of the monitoring devices described herein.

FIG. 5 shows a smart garment system. One limitation on wearable sensors can be body placement. Devices are typically wrist-based and may occupy a location that a user would prefer to reserve for other devices or jewelry, or that a user would prefer to leave unadorned for aesthetic or functional reasons. This location also places constraints on what measurements can be taken and may also limit user activities. For example, a user may be prevented from wearing boxing gloves while wearing a sensing device on their wrist. To address this issue, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware and may further enable sensing techniques that cannot be achieved with single sensing devices. For example, embedding a plurality of physiological sensors or other electronic/communication devices in a shirt may allow electrical sensors to be placed around a torso to support electrocardiogram (ECG) based heart rate measurements, or placed around muscles such as the pectoralis major, latissimus dorsi, biceps brachii, and other major muscle groups to support muscle oxygen saturation measurements. In another aspect, optical sensors may be positioned along an arterial pathway or the like to support pulse transit time measurements for calculation of blood pressure. The infrastructure provided by a garment may also support other supplemental functions beyond physiological monitoring. For example, wireless antennas may be placed above the upper portion of the thoracic spine to achieve desired communications signals, or a contactless payment system may be embedded in a sleeve cuff for interactions with a payment terminal. Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment that incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications, and so forth. The garment may also or instead include a power supply, a power management system, processing hardware, data storage, and so forth, any of which may support enriched functions for the smart garment.

In general, the smart garment system 500 illustrated in FIG. 5 may include a plurality of components—e.g., a garment 510, one or more modules 520, a controller 530, a processor 540, a memory 542, and so on—capable of communicating with one another over a data network 502. The garment 510 may be wearable by a user 501 and configured to communicate with a module 520 having a physiological sensor 522 that is structurally configured to sense a physiological parameter of the user 501. As discussed herein, the module 520 may be controllable by the controller 530 based at least in part on a location 516 where the module 520 is located on or within the garment 510. This position-based information may be derived from an interaction and/or communication between the module 520 and the garment 510 using various techniques. It will be understood that, while two controllers 530 are shown, the garment 510 may include a single inter-garment controller, or any number of separate controllers 530 in any number of garments 510 (e.g., one per garment, or one for all garments worn by a person, etc.), and/or controllers may be integrated into other modules 520.

For communication over the data network 502, the system 500 may include a network interface 504, which may be integrated into the garment 510, included in the controller 530, or in some other module or component of the system 500, or some combination of these. The network interface 504 may generally include any combination of hardware and software configured to wirelessly communicate data to remote resources. For example, the network interface 504 may use a local connection to a laptop, smartphone, or the like that couples, in turn, to a wide area network for accessing, e.g., web-based or other network-accessible resources. The network interface 504 may also or instead be configured to couple to a local access point such as a router or wireless access point for connecting to the data network 502. In another aspect, the network interface 504 may be a cellular communications data connection for direct, wireless connection to a cellular network or the like.

The data network 502 may be any as described herein. By way of example, some embodiments of the system 500 may be configured to stream information wirelessly to a social network, a data center, a cloud service, and so forth. In some embodiments, data streamed from the system 500 to the data network 502 may be accessed by the user 501 (or other users) via a website. The network interface 504 may thus be configured such that data collected by the system 500 is streamed wirelessly to a remote processing facility 550, database 560, and/or server 570 for processing and access by the user. In some embodiments, data may be transmitted automatically, without user interaction, for example by storing data locally and transmitting the data over available local area network resources when a local access point such as a wireless access point or a relay device (such as a laptop, tablet, or smartphone) is available. In some embodiments, the system 500 may include a cellular system or other hardware for independently accessing network resources from the garment 510 without requiring local network connectivity. It will be understood that the network interface 504 may include a computing device such as a mobile phone or the like. The network interface 504 may also or instead include or be included on another component of the system 500, or some combination of these. Where battery power or communications resources can advantageously be conserved, the system 500 may preferentially use local networking resources when available, and reserve cellular communications for situations where a data storage capacity of the garment 510 is reaching capacity. Thus, for example, the garment 510 may store data locally up to some predetermined threshold for local data storage, below which data is transmitted over local networks when available. The garment 510 may also transmit data to a central resource using a cellular data network only when local storage of data exceeds the predetermined threshold.

The garment 510 may include one or more designated areas 512 for positioning a module to sense a physiological parameter of the user 501 wearing the garment 510. One or more of the designated areas 512 may be specifically tailored for receiving a module 520 therein or thereon. For example, a designated area 512 may include a pocket structurally configured to receive a module 520 therein. Also or instead, a designated area 512 may include a first fastener configured to cooperate with a second fastener disposed on a module 520. One or more of the first fastener and the second fastener may include at least one of a hook-and-loop fastener, a button, a clamp, a clip, a snap, a projection, and a void.

By placing a pocket or the like in one of these designated areas 512, a position of a module 520 can be controlled, and where an RFID tag, sensor, or the like is used, the designated area 512 can specifically sense when a module 520 is positioned there for monitoring, and can communicate the detected location to any suitable control circuitry.

The garment 510 may also or instead incorporate other infrastructure 515 to cooperate with a module 520. For example, the garment infrastructure 515 may include infrastructure 515 related to ECG devices, such as ECG pads (or otherwise electrically conductive sensor pads and/or electrodes that connect to the module 520, controller 530, and/or another component of the system 500), lead wires, and the like. By way of further example, the garment infrastructure 515 may include wires or the like embedded in the garment 510 to facilitate wired data or power transfer between installed modules 520 and other system components (including other modules 520). The infrastructure 515 may also or instead include integrated features for, e.g., powering modules, supporting data communications among modules, and otherwise supporting operation of the system 500. The infrastructure 515 may also or instead include location or identification tags or hardware, a power supply for powering modules 520 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 520, and so forth. More generally, any hardware, software, or combination of these suitable for augmenting operation of the garment 510 and a physiological monitoring system using the garment 510 may be incorporated as infrastructure 515 into the garment 510 as contemplated herein.

The modules 520 may generally be sized and shaped for placement on or within one or more designated areas 512 of the garment 510. For example, in certain implementations, one or more of the modules 520 may be permanently affixed on or within the garment 510. In such instances, the modules 520 may be washable. Also or instead, in certain implementations, one or more of the modules 520 may be removable and replaceable relative to the garment 510. In such instances, the modules 520 need not be washable, although a module 520 may be designed to be washable and/or otherwise durable enough to withstand a prolonged period of engagement with a designated area 512 of the garment 510. A module 520 may be capable of being positioned in more than one of the designated areas 512 of the garment 510. That is, one or more of the plurality of modules 520 may be configured to sense data using a physiological sensor 522 in a plurality of designated areas 512 of the garment 510.

A module 520 may include one or more physiological sensors 522 and a communications interface 524 programmed to transmit data from at least one of the physiological sensors 522. For example, the physiological sensors 522 may include one or more of a heart rate monitor (e.g., one or more PPG sensors or the like), an oxygen monitor (e.g., a pulse oximeter), a blood pressure monitor, a thermometer, an accelerometer, a gyroscope, a position sensor, a Global Positioning System, a clock, a galvanic skin response (GSR) sensor, or any other electrical, acoustic, optical, camera, or other sensor or combination of sensors and the like useful for physiological monitoring, environmental monitoring, or other monitoring as described herein. In one aspect, the physiological sensors 522 may include a conductivity sensor or the like used for electromyography, electrocardiography, electroencephalography, or other physiological sensing based on electrical signals. The data received from the physiological sensors 522 may include at least one of heart rate data and/or similar data related to blood flow (e.g., from PPG sensors), muscle oxygen saturation data, temperature data, movement data, position/location data, environmental data, temporal data, blood pressure data, and so on.

Thus, certain embodiments include one or more physiological sensors 522 configured to provide continuous measurements of heart rate using photoplethysmography or the like. The physiological sensor 522 may include one or more light emitters for emitting light at one or more desired frequencies toward the user's skin, and one or more light detectors for receiving light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 522 may also or instead provide at least one of continuous motion detection, environmental temperature sensing, electrodermal activity (EDA) sensing, galvanic skin response (GSR) sensing, and the like.

The system 500 may include different types of modules 520. For example, a number of different modules 520 may each provide a particular function. Thus, the garment 510 may house one or more of a temperature module, a heart rate/PPG module, a muscle oxygen saturation module, a haptic module, a wireless communication module, or combinations thereof, any of which may be integrated into a single module 520 or deployed in separate modules 520 that can communicate with one another. Some measurements, such as temperature, motion, optical heart rate detection, and the like, may have preferred or fixed locations, and pockets or fixtures within the garment 510 may be adapted to receive specific types of modules 520 at specific locations within the garment 510. For example, motion may preferentially be detected at or near extremities, while heart rate data may preferentially be gathered near major arteries. In another aspect, some measurements, such as temperature, may be measured anywhere, but may preferably be measured at a single location in order to avoid certain calibration issues that might otherwise arise through arbitrary placement.

In another aspect, the system 500 may include two or more modules 520 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, blood pressure, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly, where packet loss, latency, and the like may present challenges to real-time processing.

The communications interface 524 may be any as described herein, for example, including any of the features of the network interface 504 described above.

The controller 530 may be configured, e.g., by computer-executable code or the like, to determine a location of the module 520. This may be based on contextual measurements, such as accelerometer data from the module 520, which may be analyzed by a machine learning model or the like to infer a body position. In another aspect, this may be based on other signals from the module 520. For example, signals from sensors, such as photodiodes, temperature sensors, resistors, capacitors, and the like, may be used alone or in combination to infer a body position. In another aspect, the location may be determined based on a proximity of a module 520 to a proximity sensor, RFID tag, or the like at or near one of the designated areas 512 of the garment 510. Based on the location, the controller 530 may adapt operation of the module 520 for location-specific operation. This may include selecting filters, processing models, physiological signal detections, and the like. It will be understood that operations of the controller 530, which may be any controller, microcontroller, microprocessor, or other processing circuitry, or the like, may be performed in cooperation with another component of the system 500, such as the processor 540 described herein, one or more of the modules 520, or another computing device. It will also be understood that the controller 530 may be located on a local component of the system 500 (e.g., on the garment 510, in a module 520, and so on) or as part of a remote processing facility 550, or some combination of these. Thus, in an aspect, a controller 530 is included in at least one of the plurality of modules 520. And, in another aspect, the controller 530 is a separate component of the garment 510 and serves to integrate functions of the various modules 520 connected thereto. The controller 530 may also or instead be remote relative to each of the plurality of modules 520, or some combination of these.

The controller 530 may be configured to control one or more of (i) sensing performed by a physiological sensor 522 of the module 520 and (ii) processing by the module 520 of the data received from a physiological sensor 522. That is, in certain aspects, the combination of sensors in the module 520 may vary based on where it is intended to be located on a garment 510. In another aspect, processing of data from a module 520 may vary based on where it is located on a garment 510. In this latter aspect, a processing resource such as the controller 530 or some other local or remote processing resource coupled to the module 520 may detect the location and adapt processing of data from the module 520 based on the location. This may, for example, include a selection of different models, algorithms, or parameters for processing sensed data.

In another aspect, this may include selecting from among a variety of different activity recognition models based on the detected location. For example, a variety of different activity recognition models may be developed, such as machine learning models, lookup tables, analytical models, or the like, which may be applied to accelerometer data to detect an activity type. Other motion data, such as gyroscope data, may also or instead be used, and activity recognition processes may also be augmented by other potentially relevant data, such as data from a barometer, magnetometer, GPS system, and so forth. This may generally discriminate, e.g., between being asleep, at rest, or in motion, or this may discriminate more finely among different types of athletic activity, such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 510 and then use this detected location to select a suitable model for activity recognition. This technique may similarly be applied to calibration models, physiological signals processing models, and the like, or to otherwise adapt processing of signals from a module 520 based on the location of the module 520. In general, determining a location of a module 520 may include, e.g., receiving a sensed location for the module 520, determining the location based on communications between the module 520 and the garment 510, determining the location based on data received from a physiological sensor 522 of the module 520, and so forth.

Once determined using any of the techniques above, the location of a module 520 may be transmitted for storage and analysis to a remote processing facility 550, a database 560, or the like. That is, in addition to the module 520 using this information locally to configure itself for the location in which it is worn, the module 520 may communicate this information to other modules 520, peripherals, or the cloud. Processing this information in the cloud may help an organization determine if a module 520 has ever been installed on a garment 510, which locations are most used, and how modules 520 perform differently in different locations. These analytics may be useful for many purposes and may, for example, be used to improve the design or use of modules 520 and garments 510, either for a population, for a user type, or for a particular user.

As stated above, the system 500 may further include a processor 540 and a memory 542. In general, the memory 542 may bear computer-executable code configured to be executed by the processor 540 to perform processing of the data received from one or more modules 520. One or more of the processor 540 and the memory 542 may be located on a local component of the system 500 (e.g., the garment 510, a module 520, the controller 530, and the like) or as part of a remote processing facility 550 or the like, as shown in the figure. Thus, in an aspect, one or more of the processor 540 and the memory 542 are included on at least one of the plurality of modules 520. In this manner, processing may be performed on a central module or on each module 520 independently. In another aspect, one or more of the processor 540 and the memory 542 are remote relative to each of the plurality of modules 520. For example, processing may be performed on a connected peripheral device, such as a smartphone, laptop, local computer, or cloud resource.

The processor 540 may be configured to assess the quality of the data received from a physiological sensor 522 of the module 520, or otherwise process data as described herein. The memory 542 may store one or more algorithms, models, and supporting data (e.g., parameters, calibration results, user selections, and so forth) and the like for transforming data received from a physiological sensor 522 of the module 520. In this manner, suitable models, algorithms, tuning parameters, and the like may be selected for use in transforming the data based on the location of the module 520 as determined by the controller 530 and/or processor 540 as described herein.

A database 560 may be located remotely and in communication with the system 500 via the data network 502. The database 560 may store data related to the system 500, such as any discussed herein, e.g., sensed data, processed data, transformed data, metadata, physiological signal processing models and algorithms, personal activity history, and the like. The system 500 may further include one or more servers 570 that host data, provide a user interface, process data, and so forth, in order to facilitate use of the modules 520 and garments 510 as described herein.

It will be appreciated that the garment 510, modules 520, and accompanying garment infrastructure and remote networking/processing resources may advantageously be used in combination to improve physiological monitoring and achieve modes of monitoring not previously available.

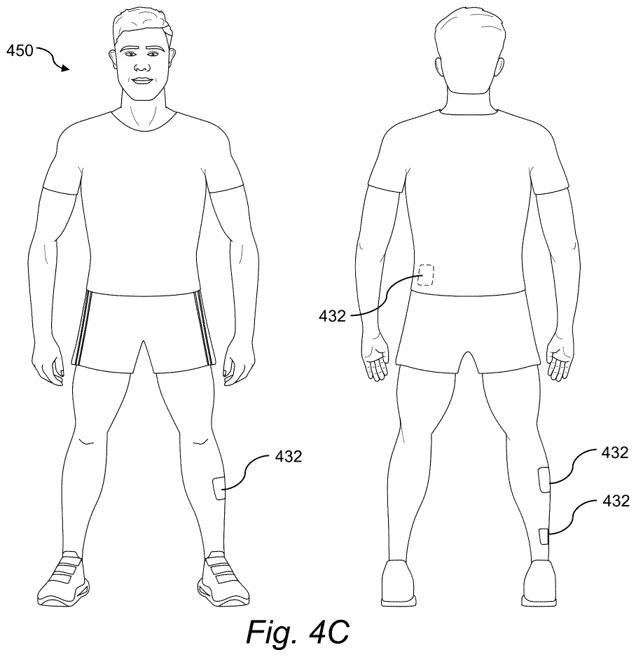

FIG. 6 shows a method for evaluating an effective age of a user based on physiological data. In general, a variety of tools may be used to correlate data from a physiological monitor, such as any of the monitors described herein, to current information available for, e.g., all-cause mortality data from academic research, in order to derive an effective age or age delta that provides a data-driven characterization of the long-term health risks associated with behaviors or characteristics. This characterization may usefully be transformed into an effective age to provide a familiar and easily comprehensible benchmark for comparison to a user's chronological age. In one aspect, the effective age calculation is based on the relationship between physiological data acquired by a wearable monitor and metrics used in reported literature concerning all-cause mortality. However, other measures may also or instead be used. For example, disease-specific mortality data may be acquired and used to create effective ages based on disease domains. Thus, a cardiovascular effective age may be derived based on cardiovascular disease mortality data, or disease incidence or risk may be used instead of mortality to achieve a measure of risk of developing a specific disease, as distinguished from a time-based conversion of risk data into an effective age. More generally, while the description herein focuses on the estimation of an effective age as an easily interpretable result for users, other results, risk scores, and evaluations may also or instead be derived for users based on the techniques described herein.

Some measurements from a physiological monitor can be directly correlated to existing research on all-cause mortality, such as movement, activity, heart rate, resting heart rate, sleep, sleep consistency, and so forth. However, even where a relatively direct comparison is available, additional challenges remain, such as double counting of individual contributions to risk, particularly when drawing on a study that does not control for other variables used in the model but not addressed in the study. Furthermore, reported data may not provide information to a user in a manner that is readily interpretable or actionable and does not generally support coaching for user-initiated behavioral modifications or any mechanism for monitoring and user feedback. As a significant advantage, the applicant's approach to measuring health provides more granular information on how multiple health metrics collectively contribute to an effective age for a user, thus facilitating improved user engagement with and understanding of the impact of behavioral modifications on health. The decomposition of an age adjustment into contributions by individual health metrics also supports the delivery of coaching recommendations that acknowledge relative strengths and target relative weaknesses. As a further advantage, behavioral recommendations can be subsequently tracked by a physiological monitor to enable status updates, dynamic coaching revisions, and user accountability.

In one aspect, a display of user health may include an age delta that shows a user's effective age compared to the user's chronological age. This data may usefully be decomposed into various components, and the contribution of each component to an aggregate effective age can then be presented to a user in order to provide the user with a lens for weighing possible behavioral interventions. A user display may also present one or more coachable variables that can be specifically measured prospectively in order to provide useful targets for a user, along with positive reinforcement, tactical recommendations, reminders, and so forth. This can facilitate monitoring of user behaviors (e.g., sleep, activity, and so forth) as well as outcomes (resting heart rate, VO₂ max, etc.) known to be associated with improved longevity. By connecting an effective age to actionable behaviors and measurable results, a user can be better coached and motivated to make changes with long-term benefits that might otherwise remain too abstract to inspire short-term behavioral modifications.

In one aspect, a behavioral modification system based on physiological monitoring can be augmented with supplemental data from other data sources. For example, blood glucose level, VO₂ max, weight, body mass index, body composition, and the like, where not directly available from a physiological monitor, can be obtained from other sources and used to augment monitoring, effective age calculations, and coaching as described herein. In another aspect, supplemental data may include measurements that, while potentially derived from physiological monitoring data, might be less accurate. Thus, for example, where an uncalibrated blood pressure, SPO2, or VO₂ max estimate is derived from optical heart rate data, this may still be useful in calculating age adjustments, although an estimate obtained in this manner might advantageously be distinguished from clinical measurements so that it can be weighted or de-weighted as appropriate in downstream calculations.

More generally, health metrics may include any measures of sleep, fitness, exercise duration, exercise intensity, biomarkers, demographics, and so forth, that provide demonstrable objective links to long-term health. This may include measurements from a physiological monitor (e.g., heart rate), measurements inferred from data from the physiological monitor (e.g., blood pressure), or data from some other source. Also, while a very broad range of characteristics are associated with long-term health outcomes, some of them may be causal and others not, and some of them may be modifiable and others not. For example, the zip code in which a user is born is very highly correlated with all-cause mortality; however, it is not causal and it is not modifiable. Thus, useful health metrics may advantageously focus on coachable metrics that provide a basis for measuring and/or inspiring behavioral change.

Data to support an effective age calculation may be acquired over any suitable interval, e.g., one week, one month, six months, or any other suitable interval or combination of intervals that balance short-term insights and long-term changes in health and wellness. In one aspect, intervals for coaching and feedback may be matched to intervals that are used to define mortality risks in published research. However, available data may be extrapolated, normalized, or otherwise processed to support concurrent use of different reported results, promote stability in report results, or otherwise adapt available data to a more positive and beneficial user experience.

A method 600 for evaluating and managing health based on these principles is now described in greater detail.

As shown in step 602, the method 600 may include acquiring data from a monitor. The monitor may, for example, include a physiological monitor such as any of the monitors described herein, or any other monitor suitable for acquiring data for use in health calculations. Thus, in one aspect, acquiring data may include acquiring data from a physiological monitor worn by a user, e.g., on a wrist, bicep, torso, finger, or other body part. The data may include any of the data described herein, such as cardiac activity data from a photoplethysmography system or other physiological monitoring system, movement data, geolocation data, temperature data, or any other data that can be acquired with a physiological monitor and used to assess user health. It will be understood that the methods described herein may also or instead be used with third-party monitoring devices, and as such, acquiring data may include retrieving physiological or other data from a cloud data resource, user device memory, data file, or other memory or data store that receives and stores data from a physiological monitoring device or other data source.

As shown in step 604, the method 600 may include calculating user metrics from the data. This may, for example, include calculating a plurality of metrics such as health metrics for the user based on the data from the physiological monitor. A variety of health metrics are known to be associated with all-cause mortality statistics and may be used as metrics for calculating an effective age as described herein.

In one aspect, the plurality of metrics may include a resting heart rate for the user, which may be based on the cardiac activity data from the physiological monitor. In one aspect, the resting heart rate may be measured during an automatically detected sleep interval for the user. By timing a measurement in this manner, e.g., during a particular phase of sleep, or during a particular time within a particular phase of sleep, or averaged over a number of episodes of a phase of sleep over the course of a night, the resting heart measurement can be repeated each day under consistent user conditions in order to facilitate more accurate inferencing and side-by-side comparison of daily resting heart rate measurements.

The plurality of metrics may also or instead include an exercise metric for the user based on a portion of the cardiac activity data recorded during an exercise activity. In this case, an intensity of exercise may usefully be estimated based on heart rate zones, activity detection, or some combination of these, and may be used to evaluate the duration and difficulty of exercise performed by a user so that this information can be used to assess how user exercise habits and trends impact an effective age.

In another aspect, the plurality of metrics may include a sleep metric, or a number of sleep metrics, derived from the data. In general, the sleep metric may include one or more of a quantity of sleep, a quality of sleep, a sleep debt, a sleep consistency, a quantity of restorative sleep, an amount of sleep relative to sleep need, or any other metric or combination of metrics for assessing the quantity and quality of user sleep. The sleep metric may also or instead include a sleep score that integrates two or more of the foregoing sleep metrics into a single, scaled score providing an overall evaluation of user sleep. In general, techniques are known in the art for detecting sleep onset, awakening, and transitions between various stages of sleep, e.g., using combinations of cardiac data, motion data, temperature data, audio data, and so forth in order to demarcate sleep events and measure the quantity and quality of sleep for use in a sleep metric as described herein.

In another aspect, the plurality of metrics may include a movement metric for the user. Motion data from a physiological monitor may be used in a variety of ways to measure the amount and type of physical activity by a user. In one aspect, the movement metric may be based on detecting steps by the user with movement data from the physiological monitor. For example, the movement metric may be based on strength training repetitions, and may characterize features such as repetitions, exercise type (e.g., based on motion signatures), strength training difficulty (e.g., based on muscle shaking), and so forth. The movement metric may also or instead include a daily step count for the user. In this case, user movements such as arm swings may be detected by motion sensors in a wearable physiological monitor and converted into a count of the number of steps taken by the wearer. In another aspect, a smartphone or the like may count steps based on in-pocket motion detection, and this may be transmitted to the physiological monitor, or to a remote data store for use in age-based evaluations and coaching as described herein. As another example, the movement metric may include a relative timing and/or sequencing of sedentary and active intervals by a user, which may usefully be correlated to research concerning the negative health impact of prolonged sedentary intervals, and/or the ameliorating effects of intermittent light or moderate activity. The movement metric may also or instead include an estimate of distance traveled, e.g., based on GPS tracking or other geolocation capabilities of the wearable monitor, or a smartphone or other accessory carried by a user, during activities such as walking, hiking, rucking, jogging, and so forth. This data may be used to estimate a distance traveled by a user, and/or elevation changes over the course of travel, which may inform an estimate of exertion by the user during such activities.

Other motion data may also or instead be used. For example, the movement metric may include an activity classification based on the movement data, e.g., using a machine learning classification model trained on motion data sets labeled for activity type. This may be used, for example, to distinguish among disparate activities such as swimming, biking, walking, or weightlifting, each of which may have significantly different strain profiles due to the nature of muscular exertion relative to detected body movement and/or measured heart rate zones. In another aspect, the movement metric may also or instead be used to infer gait metrics or the like, such as stability, step length, walking speed, and so forth, which may also or instead be used to evaluate movement and its contribution to the intensity of activity.

In another aspect, the plurality of metrics may include an exercise metric. This may be any measure of the duration, physical difficulty, and/or cardiovascular load of an activity by the user. This may include a categorization of an activity, which may be automatically detected based on data from the physiological monitor (e.g., using a machine learning classification model), or this may be manually entered by a user, or some combination of these. In one aspect, the exercise metric may include a strain score or other metric based on cardiovascular load, e.g., based on heart rate zones for one or more exercise activities by the user. For example, the exercise metric may measure a strain imposed on the user by an activity based on, e.g., a time series of heart rate reserve percentages acquired over the course of an activity, which may be averaged or otherwise normalized to provide a consistent measurement for day-to-day comparison and longer-term averaging. In one aspect, a measurement based on a series of heart rate reserve measurements may be weighted based on the heart rate zone of each sequential measurement period. In this manner, different types of aerobic activity may be weighted differently when calculating an overall strain for a particular interval of exercise. In another aspect, the exercise metric may include an average daily time within one or more target heart rate zones, and/or the time within the one or more target heart rate zones over the course of a particular exercise activity. The exercise metric may also incorporate subjective measures of exertion, which may be provided, for example, by direct user feedback received from the user after an exercise, or by a history of prior user inputs and/or strain measurements for other exercise activities. In one aspect, the exercise metric may include a calorie count or other measure of energy expenditure, which may be estimated based on data from the physiological monitor or obtained from exercise equipment. More generally, any measure of the duration, intensity, and/or type of an activity may be used as an exercise metric for calculating an effective age and other processing as described herein.

Other health metrics may, for example, include metrics that are not directly measured by a physiological monitor but can be inferred from physiological monitoring data. For example, data such as blood pressure, pulse wave velocity, and VO2 max, can be derived from photoplethysmography or other optical data and used as health metrics.

In one aspect, one or more of the plurality of metrics may be averaged over various intervals, such as at least seven days, at least thirty days, at least three months, or at least six months. In general, to obtain a stable and reliable indicator of effective age, the raw fitness measurements may be aggregated along multiple complementary time horizons, each chosen to capture a different physiological signal-to-noise profile while minimizing day-to-day volatility.

In one aspect, daily aggregation may be used. Thus, some or all of the primary fitness inputs—heart-rate zone minutes, step count, resting heart rate, VO₂ max estimates, sedentary time, and so forth—may be summarized over discrete twenty-four-hour periods. This daily snapshot neutralizes intraday spikes and ensures that the same length of biological time is always being compared. These daily averages may then be combined into a medium-term measure, e.g., a medium-term average using an unweighted moving average that spans the most recent week, two weeks, or thirty days. A month-long window is long enough to smooth out outliers caused by illness, travel, or an unusually strenuous training block, yet short enough to remain responsive to genuine behavioral changes, thus providing a useful medium-term measure of fitness. The thirty-day mean may provide a smoothed indicator of short-term health status and an early indicator of whether recent interventions (for example, a new strength routine) are making an observable impact.

In another aspect, a long-term measure of fitness status may be used. For example, for the effective-age calculation itself, a trailing one-hundred-eighty-day average may be applied to every exercise and recovery metric. Half a year of continuous data provides numerous advantages, such as capturing seasonality to account for changes in outdoor activity, sleep schedules, temperature, daylight, and the like, while limiting local bias presented by a particular season or pattern of weather, daylight, and so forth. At the same time, a long-term measure, such as six months, facilitates the capture of slower-changing trends due to physiological adaptations, such as improvements in VO₂ max, reductions in resting heart rate, or changes in weight or muscle mass, which may require weeks or months of consistent training to manifest measurable results. The longer-term window can also help to mitigate the influence of illnesses, race taper weeks, or brief lifestyle disruptions, thus preventing spurious swings in the effective-age output.

Where finer control is desired, averages, such as the six-month window, can be supplemented with an exponential decay coefficient (for example, λ=0.005 day⁻¹) so that more recent days receive slightly greater weight while still retaining information from the full period. Averaging for different metrics may be synchronized in those cases where two or more metrics are used in a single calculation. More generally, each metric may be averaged over similar or identical calendar windows before a specific hazard-ratio transformation is applied to derive an age adjustment. This can be used to mitigate temporal misalignment (due to concurrent use of short and long-term measurements) or recency bias that might create artificial changes in an effective age that are not related to corresponding changes in health and all-cause mortality risk. More generally, a tiered averaging scheme that uses different windows for different metrics can produce effective age estimates that are stable enough for year-over-year tracking and sensitive enough to register meaningful shorter-term behavioral shifts, thereby yielding an effective age that is trustworthy, interpretable, and actionable for the user.

In another aspect, the plurality of metrics may include behavioral metrics. By focusing on the age effects of behavioral metrics that can be observed by a user and/or monitored by a wearable physiological monitor, a system may advantageously provide aging information that is more easily interpretable by a user and that is more amenable to direct coaching and progress monitoring. At the same time, other descriptive metrics, such as maximal oxygen consumption, resting heart rate, body composition, or weight, may serve as better long-term indicators of actual progress toward fitness goals. In this latter category, descriptive metrics that can be directly measured by a wearable physiological monitor can advantageously support frictionless continuous monitoring and feedback that requires little or no additional effort by a user. Thus, in one aspect, a system or method as described herein may discretely separate age adjustments into one or more behavioral metrics that can be easily tracked and coached, and one or more descriptive metrics that provide stable, tangible indicators of long-term health progress.

As shown in step 606, the method 600 may include acquiring additional health measurements. In general, additional health measurements may include a chronological age of the user, a body mass index of the user, a maximal oxygen consumption for the user, and so forth. Additional health measurements may also or instead include demographic data, such as chronological age (or birth date), gender, and so forth. These additional health measurements may be obtained from a variety of sources. For example, a health measurement may be received as a manual input from a user, such as a weight, a body mass index, or any other manually entered data. In another aspect, additional health measurements may be acquired from other health measurement equipment, such as a scale for weight or body composition, energy expenditure from an exercise machine, blood pressure from a blood pressure cuff, or a maximal oxygen consumption measured at a clinical facility. It will also be appreciated that some health measurements may be inferred from other data. For example, an accurate maximal oxygen consumption measurement may be calculated for a user based on heart rate data, motion data, and other data, either alone or in combination with one or more clinical measurements. Thus, in one aspect, a metric, such as maximal oxygen consumption, may be calculated from user data acquired by a physiological monitor or may be acquired as an additional health measurement received from a user or some other resource.

As shown in step 608, the method 600 may include calculating age adjustments based on the plurality of metrics. In one aspect, this may include calculating a first plurality of age adjustments based on the plurality of health metrics (based on data from a wearable monitor) and calculating a second plurality of age adjustments based on the plurality of health data measurements (from other data sources).

As shown in step 610, the method 600 may include calculating age-related data. For example, this may include calculating an effective age of the user by adjusting the chronological age of the user according to each of the first plurality of age adjustments (based on health metrics from the physiological monitor) and each of the second plurality of age adjustments (based on other health measurements). In one aspect, the effective age may be evaluated as a series of calculations, such as calculating a plurality of age adjustments for the user based on the plurality of health metrics, calculating a total age adjustment for the user based on a weighted sum of the plurality of age adjustments, and calculating an effective age for the user by adjusting a chronological age of the user according to the total age adjustment. Other age-related data may include a pace of aging, a difference between a chronological age and an effective age, and so forth.

In one aspect, the plurality of age adjustments for the user may be based on the plurality of health metrics, where each of the plurality of age adjustments is derived from the relative risk of the individual's value for the health metric compared to the value of the health metric for a referent, such as some chosen population or benchmark. This approach advantageously facilitates the use of published and/or proprietary all-cause mortality data by transforming historical data and associated hazard ratios into effective result variables for expected longevity. For example, data from a physiological monitor can be reduced to standardized health metrics (e.g., resting heart rate, VO₂ max, heart-rate zone minutes, sedentary time, sleep duration and consistency, daily steps, etc.) and each of these metrics can be compared to a clinically grounded referent, such as public health recommendations, to obtain a metric-specific exposure level. This exposure can then be mapped to a corresponding hazard ratio derived from all-cause mortality studies that report risk as a function of the underlying metric.

Provided two assumptions are met ((1) the change in risk (i.e., hazard ratio) between the exposed and referent groups remains consistent across ages and (2) the change in the baseline hazard with respect to age is log-linear), the calculation of an effective age is straightforward and may be determined by estimating the hazard ratio associated with the behavior or condition compared to the “referent” risk, estimating the risk associated with aging for the outcome of interest, and translating the hazard ratio into an effective age difference. This is done by determining an age, t, such that e^{ct}=e^{b}, where e^{c} is the hazard ratio in the outcome of interest associated with a one-year increase in age, and e^{b} is the hazard ratio associated with the exposure. Further, for all-cause mortality, each additional year of age corresponds to a roughly 10% increase in risk. Assuming a log-linear increase in baseline mortality risk with age after early adulthood, the hazard ratio r for a given metric can be converted into an “age advancement” using a transformation such as:

$$t = \frac{ln ( {e}^{b} )}{ln ( {e}^{c} )} = \frac{ln ( H R )}{0.1} = 10 \times ln ( H R )$$

where 0.1 reflects an approximate 10% per-year baseline increase in all-cause mortality. The resulting per-metric age adjustments for each health metric can then be combined—optionally with statistically derived weights to mitigate collinearity and to avoid double counting of shared physiological effects, or having otherwise accounted for collinearity of other metrics in the derivation of the hazard ratio—into a total age delta that can be applied to the user's chronological age to yield an effective age.

It will be understood that these general conditions make a transformation into effective age relatively straightforward. However, this approach may also be modified as necessary to accommodate other hazard relationships. For example, some all-cause mortality is highly variable across ages and between genders. In other cases, all-cause mortality data has a non-uniform response to varying exposure. For example, in some cases there may be a U-shaped or parabolic relationship between exposure and risk. In other cases, there may be thresholds beyond which risk is not influenced by additional changes in exposure. In these cases, which do not provide well-behaved relationships suitable for regression or continuous mathematical modeling, a lookup table, machine learning model, or other tools or techniques may be used to provide meaningful age adjustments over a range of possible inputs.

In one aspect, the choice of referent risk may be an important factor in evaluating an effective age. The referent risk establishes the baseline against which a user's measured behaviors and physiological metrics are compared. The choice of referent therefore can control both the sign and the magnitude of each per-metric age adjustment as well as the total effective age. Thus, for example, when the referent is set to public health or physician recommendations (for example, 150 minutes of weekly exercise, 7-9 hours of sleep, age- and sex-appropriate VO₂ max and lean mass thresholds, and resting heart rate below a specified percentile), failure to meet the recommendation produces a hazard ratio greater than one and a positive age advancement, while meeting or exceeding the recommendation yields a hazard ratio at or below one and a non-positive age advancement. By contrast, if a population average is used as the referent, many users who are merely “typical” may receive neutral or favorable adjustments even when their behaviors do not meet clinically advised levels, which may result in poor coaching signals and user confusion over typical behavior versus healthy behavior.

The choice of referent also affects how metric-specific mappings are calibrated. For example, age-dependent and sex-dependent referents for VO₂ max or lean mass anchor the transformation at physiologically appropriate targets—and it influences stability over time. While guideline-based referents may change slowly, cohort-based referents can drift with membership mix or seasonality, introducing spurious shifts in effective age. In all cases, once the referent is fixed, the exposure to a behavior or characteristic can be mapped to a hazard ratio from all-cause mortality literature and converted to an age delta as described herein, so the referent selection will tend to influence or determine the zero against which relative positive and negative impacts are identified, as well as how much headroom exists for adjustments. Furthermore, the shape of any associated hazard curve may influence the sensitivity of the model to improvements or degradations in the underlying metrics.

While described sequentially for purposes of explanation, these various calculations and modeling steps may be performed in parallel or as a single calculation where possible, and some aspects of the model may be precalculated, or some combination of these. In another aspect, the individual age adjustments may be obtained using machine learning techniques or other mathematical modeling techniques. While a single effective age adjustment may also or instead be obtained in this manner, the individual adjustments on a per-metric basis advantageously facilitate coaching and progress monitoring as further described herein, and as such, may provide utility beyond their use as an intermediate result in calculating the effective age.

As shown in step 612, the method 600 may include displaying age-related data. For example, this may include displaying the effective age of the user, the chronological age of the user, or a difference between the effective age and the chronological age. In another aspect, displaying age-related data may include displaying a pace of aging of the user relative to a pace of chronological aging of the user, e.g., based on a difference between the effective and chronological ages. Displaying age-related data may also or instead include displaying a suite of age data such as the effective age, the chronological age, and a pace of aging of the user. In another aspect, displaying age-related data may include displaying each of the plurality of age adjustments and the total age adjustment applied to the chronological age. In another aspect, displaying age-related data may include displaying a health score that is calculated based on the effective age of the user and/or related data such as the chronological age, the pace of aging, and so forth.

The pace of aging may be calculated in numerous ways. For example, the pace of aging may be expressed as a difference or ratio between the effective age and the chronological age. In another aspect, the pace of aging may be calculated assuming that thirty-day averages for behavioral metrics are sustained for the next six months. This projected age may then be compared to the current effective age to determine a pace of aging. Thus, for example, if the projected effective age in six months is six months greater than the current effective age, then the pace of aging may be equal to 1. This may be particularly useful where the effective age incorporates long time spans, e.g., using data that is averaged over 3-6 months or more. In this context, the pace of aging can advantageously provide a shorter-term indicator of directionality before the current behavior manifests in the effective age. Thus, a user can receive positive feedback on improved behavioral patterns without waiting six months for these changes to fully manifest in an improved effective age calculation.

In one aspect, age-related data may be graphically presented for improved user interpretability. For example, displaying age-related data may include displaying a graphic to the user that encodes information about the effective age. In general, the graphic may be color-coded, sized, positioned, animated, or otherwise visually varied to encode age-related information. While the nature of these variations is generally a matter of visual design choice, an abstract image may advantageously employ different visual mechanisms to encode different aspects of age data. For example, in one aspect, the graphic may include a color based on a magnitude of a difference between the effective age and the chronological age. In another aspect, the graphic may include a perturbed circle with a magnitude and a number of deviations in a perimeter of the perturbed circle that increase inversely in proportion to the pace of aging. Thus, for example, a smoother, more regular shape may indicate better age metrics, while a more irregular, varied perimeter may indicate worse age metrics. In another aspect, the graphic may include an animation that varies in speed according to an age-related metric for the user. Other visual features, such as size, transparency, and overall shape, may be varied to either collectively signal the status of a single age-related figure of merit or individually to signal the status of several different metrics in a single graphic. In either case, the overall visual effect may be used to convey a generally favorable or unfavorable status in a manner that is understandable by a user.

In another aspect, displaying age-related data may include displaying each of the plurality of age adjustments derived from individual health metrics, along with the total age adjustment that is applied to the chronological age. That is, the positive and negative contributors to the age adjustment may be cataloged and presented in summary form so that a user can see which health metrics are contributing to improved and impaired risk. As a significant advantage, this approach to data presentation permits a user to identify areas suitable for focus and improvement and areas that are already well managed by the user. Where the health metrics are behavioral metrics, this also translates quickly and easily into possible remediations, which may be used, e.g., to generate coaching recommendations as described below.

As shown in step 614, the method 600 may include providing coaching recommendations. In general, this may be any recommendations, proposed activities, training regimens, schedules, and the like for the user to reduce the effective age. In general, this may include specific changes to current or historical activity patterns, new activity recommendations, and so forth. In one aspect, providing coaching recommendations may include providing coaching to the user to improve the pace of aging and/or age adjustment based on one of the health metrics.

In one aspect, coaching may include providing a fitness goal that impacts the effective age and that can be monitored for progress by the physiological monitor, e.g., via one of the behavioral or descriptive metrics described above. For example, the fitness goal may include a goal for one of the plurality of health metrics, such as a specific behavioral target for weekly time in training with a zone 4-5 heart rate, an amount of restorative sleep, or time or volume of strength training. Thus, in one aspect, the fitness goal may include a behavior used for calculating the effective age. The fitness goal may also or instead include a goal for one of the plurality of health metrics, such as a specific descriptive target. In the case of a descriptive target, such as resting heart rate, the tactical goals (e.g., other metrics, such as length and intensity of cardiovascular load) might differ, and the time horizons for measurable improvement might be longer. However, progress toward the target can still usefully be measured with a wearable physiological monitor to support dynamic coaching based on continuous monitoring and feedback. In one aspect, the fitness goal may, for example, include a descriptive target, such as a resting heart rate, a maximal oxygen consumption, and a body mass index. The fitness goal may also or instead include a behavioral target, such as a daily step count, a strength training goal, a weekly time in one or more heart rate zones, a sleep goal, and so forth. The sleep goal may include any suitable measure of the quality or quantity of sleep, such as a duration of sleep, a sleep debt, or a sleep consistency.

Coaching may be improved in a number of ways to provide a more interactive and continuous coaching experience for a user. For example, providing coaching may include displaying a plurality of fitness goals to the user in an interface. The user interface may be configured to receive a selection from the user specifying a selected fitness goal from the plurality of fitness goals and, in response to the selection, to generate a plan for the user to progress toward the selected fitness goal. The interactive coaching may include an iterative goal selection process, e.g., where a number of alternative plans are suggested for a particular fitness goal, permitting the user to choose a particular action plan. This approach promotes user agency in selecting the manner in which a goal can be pursued and permits a user to select activities most suited to a user's geography, available training resources, temperament, daily schedule, diet, and so forth. The user interface may also facilitate exploration of various alternative goals and coaching plans. In one aspect, a coaching engine that supports user coaching may be configured to generate predicted results of different training regimens so that a user can enter a tactical goal (e.g., 45 minutes of zone 3 cardio, 3 times per week) and receive a one-month, three-month, and six-month prediction of the resulting changes in one or more outcome-based health metrics. For example, the user may select a coaching plan and the user interface may present predictions for a resting heart rate, a VO2 max, a weight, a lean body mass, and so forth, after each time period, along with corresponding changes to the user's effective age.

In another aspect, recommendations or candidate recommendations for fitness goals may be sorted or filtered based on the amount of time or effort that the fitness goal will take to realize. For example, it may be easier for a particular user to add 15 minutes of zone 2 exercise than to add 30 minutes of sleep. But it may be difficult for the user to weigh these two options without information about the expected benefits of each. Thus, a recommendation may be selected or promoted based on the relative return (in terms of effective age improvement) for an amount of time or effort invested. Recommendations may also or instead be ranked directly based on difficulty for a particular user based on, e.g., a user's explicit preferences, exercise and sleep patterns, or other behavioral observations, or some combination of these.

As shown in step 616, the method 600 may include monitoring user progress toward coaching goals, such as any of the coaching goals described herein. For example, coaching may include monitoring user activity relative to one or more coaching recommendations and providing feedback to the user concerning progress toward one or more fitness goals associated with the effective age. For example, where a user has selected a goal of increasing average daily step count, the daily steps may be monitored by a wearable physiological monitor and the user may receive daily or weekly updates on how actual activity compares to fitness goals. Where the user has failed to meet a goal or is on track to fail to meet a weekly or monthly goal, the user may be notified of the deficiency and provided with suggested behavioral remediations or goal revisions. On the other hand, where a user is consistently meeting one or more fitness goals, the user may be provided with corresponding messages and feedback. Where progress toward a goal is made, this may also be explicitly reported. For example, where a user has provided a resting heart rate goal, a message may be provided indicating whether and to what extent the user is advancing toward that goal, particularly after sufficient time has passed for measurable progress to be detected. As noted above, behavioral goals may be more amenable to immediate feedback and adjustment, but either behavioral or descriptive targets may be used as the basis for interactive coaching as described herein.

More generally, monitoring user progress may include establishing a goal, e.g., for sleep or exercise, and then monitoring user activity, e.g., with a wearable physiological monitor to evaluate progress toward the goal. Monitoring progress may also include recording progress so that user behavior can be evaluated and summarized after completion of a particular coaching plan. Monitoring may also or instead include providing periodic updates, e.g., daily or weekly, so that a user can monitor the execution of a particular coaching plan. Monitoring may also or instead include providing synchronous updates, e.g., upon completion of activities such as an intense aerobic workout, a strength training session, or a night of sleep, after which a user may be informed of whether and how the completed activity has impacted the current coaching plan.

By way of a more specific example, a user may have a goal of increasing a daily step count. Progress toward a coaching goal related to the daily step count may proceed as follows: At week 0, a coaching engine and the user may interactively agree on an objective of 9,000 steps per day to address an age adjustment of +1.4 years that is driven by a low step count. A current baseline may also be established based on user data, e.g., 6,700 steps per day. During week 1, a wearable monitor may record daily steps, and a server or other processing resource may aggregate daily steps over 7 days into a mean of 7,450 steps per day. The coaching engine may monitor progress by computing a delta-to-goal (−1,550 steps) and comparing this with a prior week to identify a +750 step improvement. The coaching engine may also monitor and/or report adaptation metrics such as changes to heart rate variability or resting heart rate. If these remain within typical historical ranges, no recovery or improvement concerns may be raised during early weeks. At the beginning of week 2, an app dashboard may show progress toward a goal. In general, this may be a goal in intensity, duration, adaptive metrics, or some combination of these. In the above example, where progress (a current step count of 7,450 steps per day) from the baseline of 6,700 toward the target of 9,000 has occurred, this progress may be reported to the user as a percentage of progress toward the goal, a number of weeks of improved steps, and so forth. In addition, coaching feedback in the form of text, either within the app or as a separate text, email, or other communication, may be presented to the user, such as “Nice work-average steps increased by 11% last week. Keep the momentum: aim for 8,100 steps per day this week.”

Monitoring toward progress may also include providing interim coaching adjustments. Continuing with the above example, if progress exceeds a minimum increment (e.g., ≥500 steps), the coaching engine may advance a daily micro-target by +650 steps (10% of baseline) rather than jumping directly to 9,000. Conversely, where a user shows a poor recovery score (e.g., high resting heart rate or low heart rate variability) or poor compliance, the coaching engine may maintain or reduce the current daily target. In order to perform continuous monitoring, each night a rolling 7-day mean may be updated, and coaching may be adapted based on this metric. For example, if the mean drops by ≥750 steps or if heart rate variability falls below some historic trend or average, the coaching engine may trigger an interim remediation (“take a short walk break?”) or suggest deferring a next step increment for the following week. Conversely, if the 30-day mean reaches or exceeds the target (e.g., ≥9,000 steps per day), the goal may be recalculated and the system may select the next contributor in the severity ranking of health metrics for subsequent coaching. The user may also be notified of the beneficial results of achieving the target, e.g., “expect a reduction of effective age of about 1.4 years in the next 6 months.”

This closed-loop process—daily sensing, weekly aggregation, threshold-based adaptive targets, and periodic load checks—can usefully provide monitored coaching to ensure that a user receives timely, individualized feedback while mitigating overreach and keeping the coaching plan aligned with user objectives.

As shown in step 618, the method 600 may include other processing. This may include any other processing that might usefully be performed by a system and methods as described herein. For example, this may include archiving age-related data for historical review and evaluation of coaching effectiveness. This may also or instead include storage of coaching history and resulting behavioral changes, e.g., to develop more effective coaching plans in the future. This may also or instead include automated reminders to the user or other interested parties about proposed coaching plans, progress on existing coaching plans, and results of prior coaching plans, in order to provide users, coaches, caregivers, health professionals, and other interested parties with information to assist in fitness planning and evaluation. For example, a user may invite accountability from a friend or colleague, and the current progress may be reported to this other person to encourage adherence to an accepted coaching plan.

According to the foregoing, in one aspect, a system disclosed herein includes a physiological monitor configured to acquire data for a user, one or more processors, and a user device configured to display an effective age to the user. The one or more processors may be configured by computer-executable code stored in a memory to perform the steps of: receiving the data from the physiological monitor, calculating a plurality of health metrics for the user from the data and calculating a plurality of age adjustments for the user based on the plurality of health metrics, wherein each of the plurality of age adjustments is derived from a hazard ratio for a corresponding health metric based on an all-cause mortality rate for a population of users, calculating a total age adjustment for the user based on a weighted sum of the plurality of age adjustments, and calculating the effective age for the user by adjusting a chronological age of the user according to the total age adjustment.

The one or more processors may include processing resources on the user device, and/or the one or more processors may include processing resources of a remote server coupled in a communicating relationship with the physiological monitor and the user device. Thus, more generally, processing may be performed locally on a physiological monitor, remotely on a server or other cloud-based processing resource, locally on a laptop or other computing resource, or some combination of these.

In another aspect, a system described herein includes a physiological monitor including one or more sensors, the physiological monitor configured to acquire data for a user with the one or more sensors and transmit the data to a remote resource, a server configured to receive the data from the physiological monitor and calculate an effective age for the user as a weighted sum of a plurality of age adjustments, and a user device coupled to the server, the user device configured to receive age-related data based on the effective age from the server and display the age-related data to the user. In general, each of the plurality of age adjustments may be derived from a hazard ratio for one or more health metrics based on an all-cause mortality rate associated with the one or more health metrics. Each of the one or more health metrics may be derived from the data for the user received from the physiological monitor. A plurality of weights for the weighted sum may be statistically derived to avoid double counting of a contribution of each of the one or more health metrics to the effective age of the user.

The data may include motion data from the physiological monitor. The data may include cardiac data from the physiological monitor. The data may include geolocation data from the physiological monitor. The physiological monitor may include an item of exercise equipment, further wherein the data includes activity data from the item of exercise equipment during operation by the user. The physiological monitor may include an electronic scale and the data may include weight measurements obtained from the electronic scale while weighing the user.

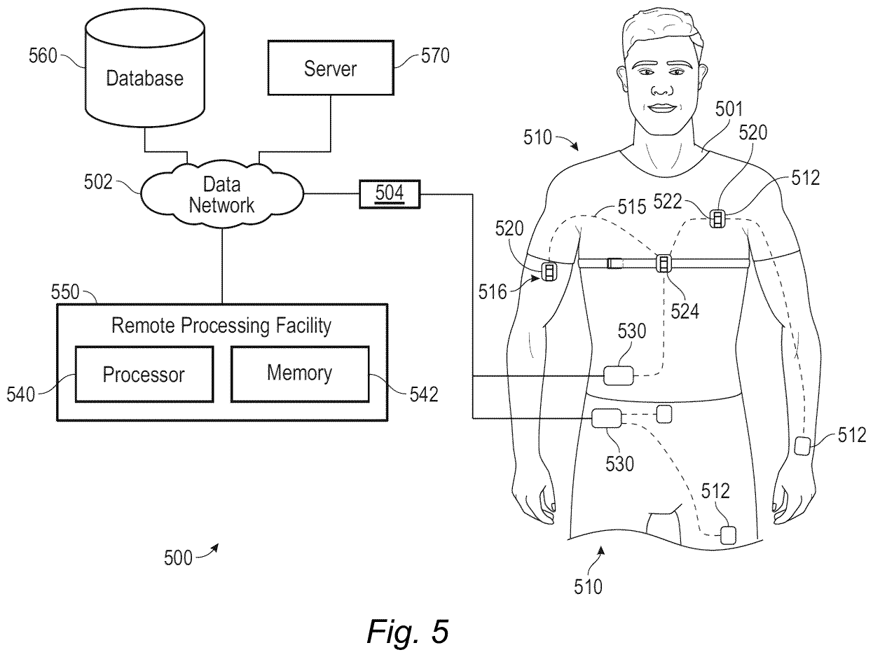

FIG. 7 illustrates a user interface for tracking effective age and other age-related data. In one aspect, the user interface 700 may display a visual summary 702 with an effective age and a comparison to the user's chronological age, along with an animation that generally provides a visual illustration of a user's relative health, e.g., with a combination of color, size, shape, animation speed, opacity, and the like. In one aspect, these independent visual components may each correlate to a different health component or group of components (e.g., sleep (e.g., duration and consistency), strain (e.g., time in heart rate zones, amount of sedentary time, strength training, etc.), or fitness (e.g., VO₂ max, resting heart rate, body mass index, and so forth)). In another aspect, various visual aspects may more generally cooperate to convey a positive or negative assessment of health relative to chronological age. It will be understood that the three separate panes illustrated in FIG. 7 may represent sequential sections of a single, continuous scrollable window that is displayed, e.g., on a phone or other user device. The user interface 700 may also or instead include a sequence of separately accessible pages. Thus, while the contents of the user interface 700 are illustrated as a single window that is separated into portions for purposes of convenience to facilitate placement in a single drawing, a variety of presentations of the user interface 700 are possible, and the user interface 700 may be adapted to, e.g., the amount of data to be presented, the device upon which the user interface 700 is rendered, and so forth. In general, the user's data may be presented in an interactive user interface so that, for any metric, age impact, or the like, a user may drill down into user history, user trends, population averages, coaching recommendations, and so forth.

For each category of metric (e.g., sleep 704, strain 706, fitness 708, and so forth), and/or each component of each category (e.g., for sleep this may include sleep consistency and hours of sleep, and for fitness this may include VO₂ max, resting heart rate, and lean body mass), the user interface 700 may display a percentile ranking or other relative or absolute measure of performance relative to a population or an individual user history, along with a positive or negative contribution by that component to the user's effective age. Thus, for example, as shown in FIG. 7, a sleep consistency may be calculated at 68%, and this may be estimated to add 1.3 years to a user's effective age. Conversely, the user may have a VO₂ max of 58 ml/kg/min, which is highly favorable and may, for the user's demographic category, subtract 5.3 years from the user's effective age. In one aspect, each metric may be interactively rendered within the user interface 700 to permit a user to access underlying physiological data, statistics, metrics, trends, population data, explanatory content, and so forth.

Other data such as an effective age trend 710 over time may be shown in comparison to the user's chronological age. The effective age trend may include a time series of data showing how a user's effective age has changed over a period of time (e.g., weeks or months) relative to the user's chronological age. A typical graph may plot effective age as one line and chronological age as another, against a shared time axis. The chronological age line will typically increase linearly with a user's monotonically increasing chronological age. The effective age line may move up or down relative to the chronological age as behavior and health metrics vary over time. When the effective age line tracks below the chronological age line and diverges further over time, this indicates that a user's long-term risks are decreasing relative to the expected long-term risks of someone who is meeting health recommendations. That is, the cumulative age deltas from sleep, activity, fitness, and sedentary exposure are net positive to health, indicating that the preexisting good behaviors are having the desired effect or that coaching is working. Conversely, when the effective age crosses above chronological age or converges toward it, the user's current habits may be eroding prior gains or not meeting recommended targets. Short, upward bumps may reflect transient disruptions (illness, travel, taper weeks), while sustained trends typically indicate real changes in behavior or physiology. Users can interpret inflection points to learn which recent changes mattered: a downward turn following improved sleep consistency, higher Zone 1-3 minutes, or more strength work suggests those interventions are effective; a flat or rising line signals the need to revisit goals (e.g., more weekly aerobic minutes, fewer unbroken sedentary bouts). In practice, one informative positive pattern is a steadily widening gap where effective age remains stably below chronological age, with only modest volatility—this indicates durable, measurable improvement in long-term health risk.

In one aspect, the user interface 700 may include a pace of aging trend 712 that shows a trend in the pace of aging over time. In general, the pace of aging is a short-horizon indicator (relative to effective age) that describes how quickly a person's effective age is changing relative to calendar time based on recent behaviors and physiological trends. Practically, the pace of aging may be computed by projecting the effective age forward using near-term averages of coachable metrics (for example, the most recent 30-day means for sleep, activity, steps, sedentary time, strength work, resting heart rate, and VO₂ max) and comparing that projection to the current effective age over a fixed interval such as six months. If the projected effective age increases by roughly the same amount as chronological age over that interval, the pace of aging is near 1. If the effective age is projected to rise faster than calendar age, the pace exceeds 1 (accelerated aging), and if effective age is projected to rise more slowly or decline, the pace is below 1 (decelerated aging or age reversal). Because the pace of aging emphasizes recent behavior, this metric will be more responsive than the six-month inputs used for effective age, providing timely feedback and actionable coaching while longer-term adaptations (e.g., VO₂ max, resting heart rate) accumulate.

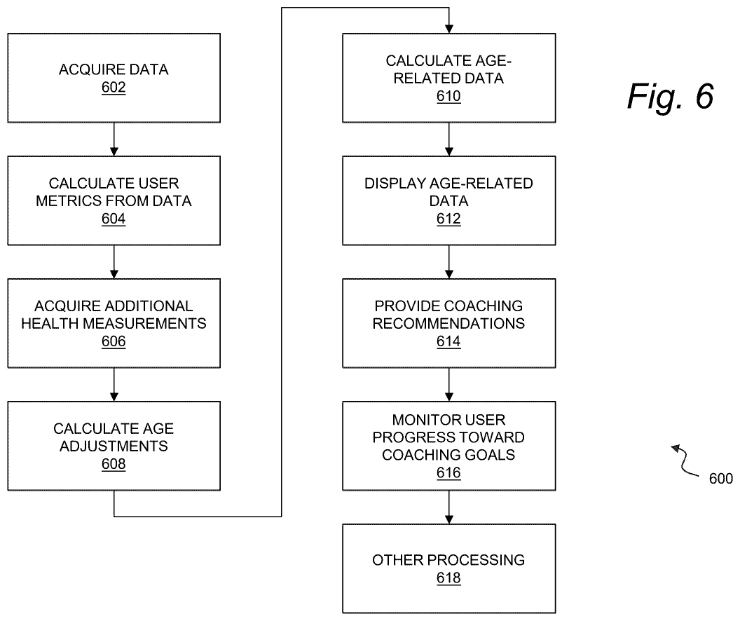

FIG. 8 shows health recommendations for a number of health metrics. In general, these recommendations 800, and the more detailed underlying statistics, provide a framework for evaluating effective age and for providing coaching around data for a particular user. It will be understood that the guidelines for health recommendations change over time with research and improvements to medical/scientific understanding of physiological causation, risk factors, contributors to long-term health, and so forth. Thus, the recommendations in FIG. 8 should be understood as illustrative of the manner in which referent behaviors and exposure can be specified for purposes of calculating age adjustments as described herein, rather than as requirements for calculating an effective age.

One fitness measure of interest is sleep duration. Adequate sleep is important for long-term health, with both insufficient and excessive sleep linked to increased health risks. A meta-analysis with over 5 million participants found that short sleep duration was associated with a 12% higher risk of all-cause mortality, alongside an increased risk for conditions such as diabetes, hypertension, cardiovascular disease, coronary heart disease, and obesity. Other studies have shown that the risk of all-cause mortality associated with short sleep is even higher when sleep duration is measured objectively using a wearable device instead of relying on self-reported measures. Given the elevated risk of long-term health consequences, users averaging less than 7 hours of sleep should see an increase in effective age. Some studies suggest that 7 hours is the optimal sleep duration, as sleep durations greater than 7 hours have also been associated with higher mortality risk. However, unlike short sleep, the biological mechanisms underlying this association are unclear. Some researchers hypothesize that prolonged sleep may be a symptom rather than a cause of poor health, potentially linked to underlying comorbidities, depressive symptoms, or disease-related fatigue. In support of this hypothesis, a study of apparently healthy individuals found that those reporting 8-9 hours of sleep per night had a slight, though not statistically significant, reduction in all-cause mortality compared to those sleeping 7 hours. As a result, a healthy recommendation for sleep may be established as 7-9 hours of sleep per night, with users in this range seeing a reduction in their effective age, while users who sleep more than 9 hours per night may have no effect on their effective age, ensuring that the metric remains evidence-based and avoids conflating correlation with causation.

Against this backdrop, sleep duration may be measured as the total sleep time a user obtains during a nocturnal sleep episode inferred from continuous physiological monitoring. In an aspect, a wearable physiological monitor may detect sleep onset and wake events using heart rate, motion, and related signals, and may calculate sleep duration as the interval between detected sleep onset and final awakening while excluding brief awakenings, thereby distinguishing total sleep time from time in bed. In another aspect, sleep duration may be aggregated over discrete twenty-four-hour periods to yield daily values, which may then be averaged over a rolling thirty-day window for pace-of-aging projections and over a trailing one-hundred-eighty-day window for effective-age calculations, optionally with an exponential decay weighting so that more recent days receive greater weight. In an implementation, sleep duration may be compared to a guideline-based referent such as at least about seven hours per night, with values below the referent mapped to a hazard ratio from all-cause mortality literature and converted to an age advancement using a transformation as described herein. Similarly, values at or above the referent may yield a non-positive age adjustment.

In this context, coaching for sleep may be configured to promote incremental, sustainable increases in sleep duration while preserving recovery and adherence. In one aspect, weekly plans may select sleep duration as a coaching target when the thirty-day average is materially below the referent or when the associated age adjustment exceeds a severity threshold. A coaching plan may set a weekly sleep-performance goal that corresponds to a small, achievable increase in expected sleep time (for example, on the order of ten to twenty minutes per night), and may couple this with behaviors that increase total sleep time: allocating sufficient time in bed by establishing a consistent bedtime and wake-time range; viewing morning sunlight to anchor circadian timing; limiting caffeine at least about fourteen hours before planned bedtime; avoiding bright screens and intense light after sundown; finishing the last meal at least two hours before bedtime; and/or reserving the bed for sleep. In another aspect, a coaching plan may recommend limiting late-day high-intensity exercise so that Zone 4-5 sessions finish at least one hour before bedtime, thereby reducing physiological arousal that may truncate sleep. Progress may be tracked weekly using aggregated sleep duration and complementary adaptation measures such as resting heart rate, heart rate variability, and subjective sleep quality; when these measures indicate overreach or poor recovery, the plan may temporarily reduce late-day training load or expand the bedtime window to facilitate additional sleep. As the user's thirty-day average approaches or exceeds the referent, coaching may shift to the next most impactful contributor to effective age, while continuing to monitor sleep duration to ensure it remains at or above the guideline threshold and does not regress due to travel, illness, or lifestyle changes.

Another health metric of interest is sleep consistency. The National Sleep Foundation emphasizes that consistent sleep-wake schedules are crucial for optimal health and performance, as irregular sleep disrupts circadian rhythms, leading to adverse health outcomes. Similarly, a prospective cohort study involving over 60,000 participants found that individuals with higher sleep regularity had a 20% to 48% lower risk of all-cause mortality, a 16% to 39% reduction in cancer mortality, and a 22% to 57% decrease in cardiometabolic mortality compared to those with the most irregular sleep patterns.

Sleep consistency may be measured as the regularity of a user's sleep and wake times across consecutive days. The system may, for example, use a sleep regularity index that is calculated as a probability that the user is in the same state (asleep or awake) at matching clock times 24 hours apart, or any other sleep consistency score that provides a consistent and objective measure of how consistent a user's sleep patterns are. In one aspect, a rolling four-day window may be used for assessing consistency, although any other window, weighted average, or the like may be used, provided it establishes a consistent measure that can be compared to a baseline or referent, and that can be compared to prior and future values for the user. A wearable monitor may detect sleep onset and wake events from continuous physiological signals (heart rate, motion, and related features) and compute a daily sleep/wake profile. These daily profiles can then be compared at fixed times to determine how often the user's state matches on successive days. In a typical sleep consistency metric, the metric is a figure of merit where a higher score indicates more favorable sleep patterns. For effective age, the system may average sleep consistency over longer windows (e.g., 30 days for pace-of-aging feedback and 180 days for effective-age calculations) and compare these results to a guideline-based referent (e.g., at least about 70% consistency, which roughly corresponds to keeping sleep onset and wake times within a one-hour window most days). Values below the referent may produce a positive age adjustment via a hazard-ratio mapping and values at or above the referent may produce a non-positive adjustment.

Coaching for sleep consistency may focus on incremental, sustainable improvements that move the 30-day average toward the referent while maintaining recovery and adherence. Weekly plans may include sleep consistency when consistency is a material detrimental contributor to effective age, prioritizing it ahead of lower-impact factors. The user may be given a target wake-time range and bedtime range (for example, tightening by 15 minutes per week) and simple, high-yield behaviors to reinforce circadian regularity: view morning sunlight shortly after waking; avoid bright screens and intense light after sundown; finish high-intensity exercise at least an hour before bed; complete the last meal at least two hours before bedtime; limit late caffeine and alcohol; and reserve the bed for sleep. Progress may be tracked with weekly aggregation (rather than day-to-day fluctuations), and adaptation status (e.g., trends in HRV, resting heart rate, and sleep quality) may be used to adjust the plan if signs of overreach or poor recovery emerge. As the user's sleep consistency crosses severity thresholds (e.g., from severe to moderate to mild contribution to effective age), coaching may shift to the next most impactful contributor, while continuing to monitor sleep consistency to ensure it remains at or above the referent level.

Another health metric of interest for coaching is heart rate zone time, e.g., the amount of weekly time spent in various heart rate zones. Regular physical activity is an effective way to improve heart health, metabolic function, and longevity. Research shows that individuals who spend more time in moderate-to-vigorous intensity exercise experience lower risks of cardiovascular disease, type 2 diabetes, cognitive decline, and all-cause mortality. Studies have reported a 20% to nearly 50% reduction in all-cause mortality with 5-7 hours of weekly physical activity, with objective activity tracking (e.g., from wearables) showing even stronger associations. Notably, even short bursts of high-intensity activity can provide substantial health benefits. One study found that just 1-2 minutes of vigorous exercise incorporated into daily routines—accumulating 15 to 20 minutes per week—was associated with an 18-24% lower risk of all-cause mortality compared to no vigorous activity. Health organizations, including the CDC and the WHO, recommend at least 150 minutes of moderate-intensity or 75 minutes of vigorous-intensity exercise per week. However, evidence suggests that additional activity provides even greater benefits, with all-cause mortality risk decreasing incrementally with up to 600 minutes per week. Similar to other metrics, research suggests that the longevity benefits of physical activity become even greater as people age, underscoring the importance of maintaining an active lifestyle.

Heart-rate zones may be measured as contiguous ranges of the user's instantaneous heart rate expressed as a heart rate reserve (HRR) relative to a resting heart (HRᵣₑₛₜ) and maximal heart rate (HRₘₐₓ). In general, the maximal heart rate may be an age-adjusted maximal heart rate estimate or a maximal heart rate estimate based on user monitoring. In one implementation, a wearable optical sensor may stream beat-to-beat intervals (with on-device or server-side smoothing), identify artifacts, and convert each epoch (for example, 1-s or 5-s bins) into a percentage of HRR, where:

$$\% HRR = \frac{( H R - H {R}_{rest} )}{( H {R}_{max} - H {R}_{rest} )} \times 100$$

Pre-defined thresholds may delineate aerobic zones of interest. While these zones are generally categorized from zone 1 to zone 5, these may usefully be combined into broader categories for purposes of evaluating effective age as contemplated herein. For example, the predefined thresholds may include Zones 1-3 (≈50-80% HRR) for light to moderate exercise and Zones 4-5 (>≈80% HRR) for vigorous/anaerobic exercise. The system may accumulate the number of seconds or minutes spent within each zone during logged exercise bouts and, separately, across the entire day. Daily zone times may be stored locally, synchronized to the cloud, and then averaged over rolling 30-day and 180-day windows to support calculations of age adjustments and other age-related data as described herein.

Coaching on heart-rate-zone exposure may follow a tiered, weekly-planning workflow. For example, when the 30-day mean of Zone 1-3 time falls below a referent, such as ≈70 minutes per week, the engine may flag moderately intense activity as a goal and propose a micro-target increase of roughly 10% (or a minimum of 5 min) for the coming week, or some other relative adjustment based on historical activity. Similarly, if Zone 4-5 time averages less than ≈3 minutes per week, a vigorous intensity goal may be established. A coaching plan for vigorous activity may include behavioral supports—hydration, stretching, morning scheduling—and activity suggestions (brisk walking, cycling, interval runs, swimming). A polarized distribution goal (≈80% Zone 1-3, ≈20% Zone 4-5) may also be recommended when total load is adequate but intensity balance is suboptimal.

Progress may be monitored with nightly updates of 7-day moving sums for each zone. If the user meets or exceeds a micro-target while remaining within adaptation bounds (e.g., HRV and resting HR within smallest-worthwhile-change thresholds), the target for the next week may be advanced by another 5-15%. If recovery markers deteriorate or the user falls short of the interim goal, the system may maintain or slightly reduce the target, deliver reminders (e.g., “add a 15-min low-intensity walk”, or schedule a deload week). Once the 30-day mean consistently meets or exceeds the referent, a corresponding age adjustment may be recalculated (moving toward zero or negative), and coaching attention may pivot to the next highest contributor to effective age.

Another health metric of interest is strength training. Engaging in regular strength training is strongly linked to reduced risks of all-cause mortality and chronic diseases, making it a key component of long-term health. Meta-analyses synthesizing findings from large cohort studies have found that performing 30 to 60 minutes of muscle strengthening activities per week is associated with a 10% to 30% reduction in all-cause mortality, cardiovascular disease, and cancer. Notably, there appears to be a U-shaped association between strength training time and all-cause mortality, with the optimal benefit from strength training time occurring between 30 minutes and 2 hours, though the mechanisms underpinning the attenuated risk reduction with larger amounts of strength training time are not well understood. As such, in certain embodiments, logging more than 2 hours per week of strength training time may optionally be ignored when performing age-related calculations.

In general, strength-training activity may be measured as the total weekly time a user spends performing predefined muscle-strengthening modalities and logged sessions. In one aspect, a wearable monitor and companion app may classify activities as strength-building (e.g., weightlifting, resistance circuits, functional fitness, Pilates, yoga variants focused on muscular load, barre), and may accumulate minutes from tagged workouts, auto-detected sessions, or equipment integrations. Session boundaries may be inferred from motion signatures, heart-rate patterns, and user confirmations (e.g., manual inputs); the resulting daily minutes may be aggregated to weekly totals, then averaged over rolling thirty-day and trailing one-hundred-eighty-day windows to supply short-term coaching (pace of aging) and long-term effective-age calculations, respectively. A guideline-based referent may be used (for example, at least about 40 minutes per week of muscle-strengthening activity, with diminishing returns beyond roughly 60 minutes per week), and minutes below the referent may be mapped to a hazard ratio and converted to an age advancement. Any minutes at or above the referent may yield a non-positive adjustment.

Coaching may prioritize incremental, sustainable increases in weekly strength training time while maintaining recovery and adherence. In one implementation, when the user's thirty-day mean falls below the referent or when the associated age adjustment exceeds a severity threshold, the system may schedule a strength monitoring statistic and propose a micro-target increase (for example, +30 minutes per week until the thirty-day mean reaches the referent). Plans may include behavior supports—adequate protein intake, hydration, and optional creatine; warm-up and stretching; and movement preparation—and activity options such as “Strength Trainer,” weightlifting, functional fitness, barre, yoga/Pilates. Progress may be monitored with nightly updates of rolling 7-day totals. If the user meets or exceeds the micro-target while recovery markers (e.g., resting heart rate and heart-rate variability within smallest-worthwhile-change thresholds) remain favorable, the next week's target may advance by 10-15%. If recovery deteriorates or the user falls short, the plan may maintain or slightly reduce the target, add pushed reminders (“short supplemental session?”), or schedule a deload week. Once the thirty-day mean consistently meets or exceeds the referent, the strength-related age adjustment may be recalculated (moving toward zero or negative), and coaching may pivot to the next highest contributor to effective age, while continued monitoring helps ensure the user sustains adequate weekly strength exposure without injury or overreach.

Another health metric of interest is a user's daily step count. Among various measures of activity, daily step count is a simple yet powerful indicator of movement and long-term health. Research consistently links higher step counts to lower risks of all-cause mortality and cardiovascular disease. A meta-analysis of 16 publications found that for every additional 1,000 steps per day, the risk of all-cause mortality decreased by 23%, while an increase of 500 steps per day reduced the risk of cardiovascular events by 6%. Research suggests that long-term health benefits of daily steps begin to plateau around 8,000 to 10,000 steps per day. As a result, younger users who exceed 8,000 steps per day may only see modest reductions in their effective age, while those falling below that threshold may have years added to their effective age, reflecting the health risks associated with lower activity levels. Additionally, research suggests that a higher daily step count may have an even greater impact in older adults. For example, a study found that for adults over 60, the same number of daily steps resulted in a greater reduction in all-cause mortality compared to those under 60, thus fewer steps may be needed to decrease the effective age of users older than 60. This reflects the importance of maintaining regular movement at all ages, particularly later in life when physical activity can provide even greater health benefits. Further, this illustrates the different risk profiles associated with different health metrics, where in some cases, there may be substantial age-dependent variations in responsiveness.

Daily step counts may be measured as the total number of steps accumulated within a twenty-four-hour period inferred from motion sensing, either by a wearable physiological monitor or an associated device such as a smartphone, smart watch, or other wearable or portable device. In one aspect, a wearable physiological monitor may use a wrist-mounted accelerometer and gyroscope to detect footfall signatures and arm-swing patterns, apply filtering to remove artifacts, and count steps using modeled gait events. Step counts may be validated or supplemented with smartphone motion data when available, and daily totals may be stored locally and synchronized to a cloud service. In another aspect, step counts may be aggregated into rolling seven-day and thirty-day means for short-term feedback, and into trailing one-hundred-eighty-day averages for effective-age calculations, optionally with exponential decay weighting to emphasize recent behavior.

Coaching may be configured to promote incremental, sustainable increases in average daily steps while maintaining recovery and adherence. In one implementation, when the thirty-day mean falls materially below an age-adjusted referent (for example, about 8,000 steps per day for mid-life adults, with lower thresholds for older adults), the system may schedule a steps coaching plan and propose a micro-target increase (for example, +500 steps per day for the coming week). The plan may pair activity suggestions—short walk breaks, post-meal strolls, staircase substitutions, longer errands on foot—with behavioral supports such as morning sunlight exposure (to reduce sedentariness), calendar prompts during known low-movement periods, and social commitments (walk with a friend). Progress may be monitored with nightly updates to a rolling seven-day mean. If the user meets or exceeds the micro-target while adaptation markers (e.g., resting heart rate or heart-rate variability) remain favorable, the next week's target may advance by another 500-1,000 steps. If recovery deteriorates or the mean drops beyond its smallest worthwhile change threshold, the system may maintain or slightly reduce the target and deliver reminders. Once a thirty-day mean for daily step count consistently meets or exceeds the referent, the step-related age adjustment may be recalculated (moving toward zero or negative), and coaching may pivot to the next highest contributor to effective age, while continued monitoring helps ensure the user sustains adequate daily movement without overreach.

VO₂ max is another health metric of interest. VO₂ max is a measure of cardiorespiratory fitness and is the maximum amount of oxygen that an individual can utilize during intense or maximal exercise, measured in milliliters per kilogram per minute (mL/kg/min). This value reflects how efficiently your respiratory and cardiovascular systems can deliver oxygen to muscles during periods of maximum intensity exercise, providing a holistic view of your fitness level. VO₂ max can range from below 20 mL/kg/min in untrained or older individuals to above 80 mL/kg/min in elite athletes.

In general, VO₂ max is a strong predictor of long-term health and all-cause mortality, reflecting overall cardiovascular and respiratory fitness more effectively than traditional risk factors like smoking, hypertension, or BMI. With even modest improvements, such as moving from the bottom quartile for VO₂ max to the 50th-75th percentile, mortality risk decreases by over 60%. Similarly, studies show a 13% lower mortality risk for every additional 1 MET (metabolic equivalent, ~3.5 mL/kg/min) increase in VO₂ max. While VO₂ max indicates a capacity at maximum intensity, a higher VO₂ max makes all daily activities easier, underscoring the importance of VO₂ max for independent daily living. VO₂ max naturally declines by 10-12% per decade after age 30, driven by factors like reduced cardiovascular capacity and changes in lean body mass. Maintaining or improving VO₂ max through regular activity and exercise mitigates these age-related declines. While there are not clear clinical or public health recommendations for VO₂ max, recommendations can be designed to target functional values at advanced ages, e.g., to account for the natural decline in VO₂ max with age. Research suggests that a minimum of approximately 20 mL/kg/min is required to maintain independent living, with males exhibiting roughly 20% higher VO₂ max compared to females. Thus, users with a VO₂ max greater than an age-adjusted and sex-adjusted target may see years subtracted from an effective age, while a VO₂ max lower than the target may see years added.

VO₂ max may be measured as an estimate of maximal oxygen uptake (ml·kg⁻¹·min⁻¹) derived from sensor data for a wearable monitor, and/or obtained from clinical testing. In one aspect, a gold-standard laboratory assessment may use indirect calorimetry during a graded treadmill or cycle protocol to exhaustion, quantifying breath-by-breath gas exchange to determine maximal uptake. In another aspect suitable for continuous monitoring, a wearable physiological monitor may estimate VO₂ max from passively collected signals—resting heart rate, heart-rate response during free-living runs or walks, pace and grade from GPS/barometer, and user demographics (age, sex, body mass index). A model may align the relationship between heart-rate and speed during steady aerobic efforts to a validated reference curve and, after artifact rejection and calibration, output a weekly VO₂ max estimate. These estimates may be aggregated over rolling thirty-day windows for pace-of-aging feedback and trailing one-hundred-eighty-day windows for effective-age calculations, optionally with exponential decay weighting to emphasize recent sessions.

Coaching may be configured to increase VO₂ max through a balanced progression of aerobic volume and high-intensity work. In one implementation, when the thirty-day VO₂ max mean falls below an age- and sex-adjusted referent, the system may schedule two complementary tracking statistics: (i) accumulate ≥90-100 minutes per week in heart-rate Zones 1-3 to build aerobic base, and (ii) accumulate ≥25-30 minutes per week in Zones 4-5 via intervals to raise the ceiling. A polarized distribution (≈80% Zones 1-3, ~20% Zones 4-5) may be recommended. Weekly micro-targets may increase by about 5-15% as long as adaptation measures (e.g., thresholds for resting heart rate and heart-rate variability) remain favorable. Activity options may include interval runs (e.g., 4-6 repeats of 2-4 minutes at Zone 4 with equal recoveries), tempo segments just below lactate threshold, long easy runs or rides (30-90 minutes), and low-impact alternatives (cycling, swimming) to manage musculoskeletal load. Progress may be monitored by tracking (i) zone minutes, (ii) the heart-rate-speed curve on repeatable routes, and (iii) the weekly VO₂ max estimate; if recovery deteriorates, the plan may insert a deload week (reduced volume and intensity) or shift intervals to lower impact. As VO₂ max rises toward or above the referent, coaching may pivot to maintenance (stable aerobic volume, periodic quality sessions) while continuing to guard against detraining—e.g., with reminders after two weeks without vigorous efforts, given that VO₂ max may decline measurably after short inactivity.

Resting heart rate (RHR) is another metric of interest. In general, a higher RHR is associated with increased risks of all-cause mortality and cardiovascular diseases like coronary artery disease and stroke. One study found that men with an RHR of 90 beats per minute (bpm) or higher had a three-fold increase in the risk of premature death compared to those with an RHR of 50 bpm or lower. While a RHR between 60 and 100 bpm in a clinical setting is considered normal, even small differences in resting heart rate, particularly when captured during sleep, are associated with meaningful differences in long-term health risk. For example, a 10 bpm increase in RHR has been associated with a 9% increase in mortality risk, underscoring the importance of maintaining a lower RHR through lifestyle and physical activity. Thus, in general, an average RHR of ~60 bpm for males and ~64 bpm for females may result in a neutral impact to effective age, while higher values will increase the effective age and lower values will decrease it.

Resting heart rate may be measured as the average beats per minute during low-arousal states inferred from continuous physiological monitoring, e.g., during slow wave sleep and/or deep sleep. In one aspect, a wearable optical sensor may capture beat-to-beat intervals overnight. Firmware may weight segments with stable autonomic tone (for example, later portions of sleep and slow-wave sleep) and apply a correction factor as appropriate to approximate daytime resting values. In another aspect, multiple nightly measurements may be aggregated to daily values and then averaged over rolling thirty-day and trailing one-hundred-eighty-day windows (optionally with exponential decay weighting) to supply short-term feedback (pace of aging) and long-term effective-age calculations. A guideline-based referent (for example, sex-specific thresholds near the lower clinical percentiles) may be used so that values above the referent map to hazard ratios and positive age advancement via a transform as described herein.

Coaching may be configured to lower resting heart rate through aerobic conditioning, recovery hygiene, and lifestyle supports. In one implementation, when the thirty-day mean exceeds the referent or when the associated age adjustment crosses a severity threshold, the system may schedule a coaching plan with complementary tracking statistics, e.g., (i) accumulate ≥90-100 minutes per week in heart-rate Zones 1-3 to improve stroke volume and parasympathetic tone; (ii) accumulate ≥25-30 minutes per week in Zones 4-5 to raise cardiorespiratory capacity, as tolerated; and (iii) improve sleep consistency and duration (e.g., ≥70% consistency and ≥7 h per night). Behavioral supports may include hydration, adequate protein, and optional creatine for training sessions; circadian anchors (morning light, reduced late caffeine and alcohol); and stress-modulating practices (brief breathwork, low-intensity evening walks). Progress may be monitored with weekly aggregation of resting heart rate alongside adaptation markers such as heart-rate variability, where if HRV falls below a predetermined threshold or RHR rises, the plan may maintain or slightly reduce training load and increase recovery emphasis. As the user's thirty-day mean approaches or crosses the referent, the resting-heart-rate age adjustment may be recalculated (moving toward zero or negative), and coaching may pivot to the next most impactful contributor to effective age while continuing to guard against regression during travel, illness, or lifestyle changes.

Lean body mass is another metric of interest. Higher lean body mass percentage is strongly associated with lower mortality risk, even after accounting for other factors like body mass index. A study of nearly one million adults across 35 cohorts found that with each 10% increase in body fat, the risk of all-cause mortality increases by 11%. The recommended ranges may vary by age and gender. Thus, for example, higher lean body mass percentages may be recommended for younger individuals and males. The recommendation for a 30-year-old female may be at least 67% and for a 30-year-old male may be at least 80%. While it may be difficult to directly measure lean body mass with a wearable physiological monitor, this data may be obtained from a connected smart scale or manually entered by a user. Users with lean body mass percentages below the referent thresholds may see years added to their effective age, while users above these thresholds may see reductions in their effective age. If lean body mass data is not available, the age adjustment may default to zero unless/until body composition and other demographic data are provided.

It will be appreciated that coaching as described herein may be delivered in a number of ways. In one aspect, the user may initially be presented with a number of options for improvement and be permitted to select a particular area for focus. Thus, the user may choose to focus on sleep instead of Zone 4-5 training. The duration of a coaching plan may be fixed or may vary based on results/progress, either in absolute terms or relative to other monitored metrics. In another aspect, coaching may focus around a particular metric of interest over an extended period of time or may dynamically adjust to the weakest metric for a user as the user's fitness evolves. For example, where a user is being coached for sleep and the user's sleep has improved to the point where a sleep-related age adjustment is less than or equal to zero, coaching may automatically change to another metric for coaching, or the user may be prompted to ask if the user would like to change focus. At the same time, coaching revisions may be tempered to avoid frequent task switching that might hinder user engagement or progress. In another aspect, a user may be presented with potential coaching outcomes while offering the user several alternative coaching plans. Thus, the user may receive context about the demands of a number of coaching plans and the likely outcomes of a number of coaching plans so that the user can make an informed decision about whether and how to pursue an improved effective age in a manner that conforms to the user's available time, level of interest and ability, and desire for progress.

This general strategy employs monitoring and feedback to promote greater user engagement and facilitate continuous, dynamic adaptation so that coaching recommendations and user behavior can both meaningfully respond to objectively measured progress. Thus, coaching plans may usefully create a feedback loop where specific targets are selected along with corresponding tracking metrics, and insights from tracking metrics can inform progress and support dynamic adjustments to a current coaching plan. Coaching can use continuous benchmarking, intervention, reinforcement, progression, assessment, and readjustment as parts of a closed-loop process around effective age and the health metrics that contribute thereto. Continuous benchmarking may include recalculating rolling 7-day, 30-day, and 180-day averages for key metrics (e.g., steps, sleep, HR-zone minutes, resting heart rate, VO₂ max) and comparing these to both the user's own historical baseline and guideline-based referents. Intervention may occur when a metric is materially worse than its referent or contributes materially to a positive age delta: the system may select a health metric as the current focus, define one or more tracking metrics (e.g., “+500 steps per day,” “+10 minutes Zone 1-3 per week”), and generate a concrete weekly plan. Reinforcement and progression may rely on weekly aggregation: when the user meets or exceeds a target without adverse adaptation signals (e.g., HRV and resting HR remain within expected bounds), the plan may display progress (charts, age-delta projections, encouraging copy) and advance the target by a modest increment (for example, 5-15% more steps or minutes) so that the difficulty scales with success. Assessment and readjustment may occur at regular checkpoints (for example, weekly and monthly), where a coaching engine re-evaluates both target metrics (e.g., effective age, pace of aging, per-metric age deltas) and measures of adaptation. If a target is repeatedly missed or physiology shows signs of overload, the plan may be maintained, simplified, or temporarily de-loaded, and a different contributor may be prioritized. Over time, this loop permits individualized, data-driven coaching that responds to actual behavior and physiological response rather than static prescriptions.

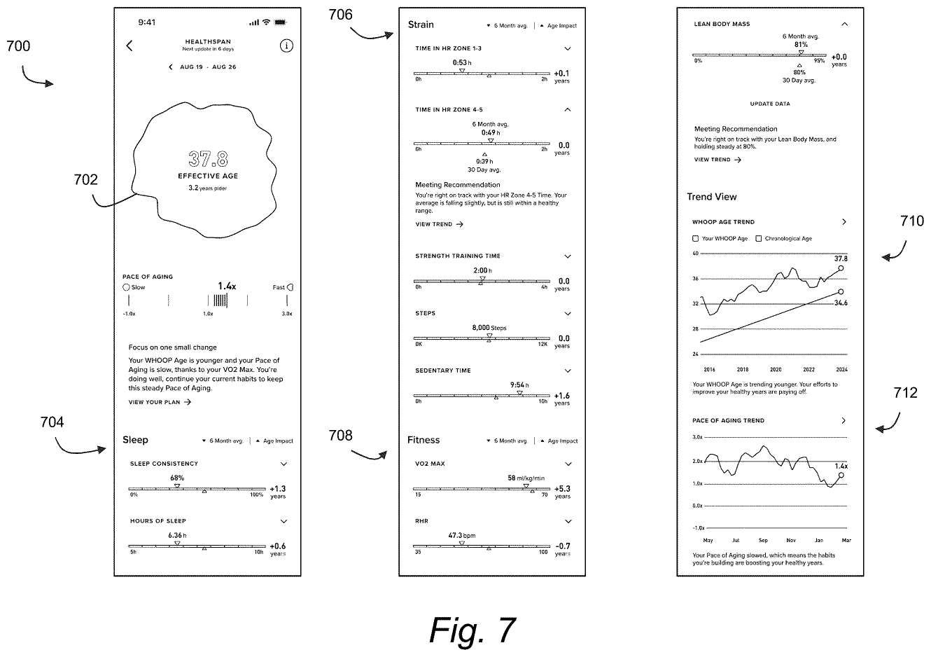

FIG. 9 illustrates an effective age calculation. As described herein, the effective age calculation may proceed by first deriving per-metric age adjustments from standardized, six-month-averaged fitness and behavior inputs, and then summing these adjustments to offset the user's chronological age. In one aspect, each input—such as VO₂ max, resting heart rate, body mass index, sleep duration, heart-rate zone minutes (Zones 1-3 and 4-5), and daily sedentary time—may be compared to a guideline-based referent and the user's exposure relative to that referent may be mapped to an all-cause-mortality hazard ratio, which may then be converted using an age advancement transform where positive values indicate metrics worse than the referent (adding years) and negative values indicate metrics better than the referent (subtracting years). It is also possible to provide a more complex or interdependent aggregation where there are interaction effects between metrics that cause multiple age effects to change in response to a single metric change. While this may impair user interpretability and consistency, e.g., where an observed change in one metric causes the age effect of another metric to change, interdependent age adjustments can also potentially support better conformity to actual hazard ratio data and provide components of age adjustment that more accurately reflect the relative contribution of each health metric. In another aspect, weights may optionally be applied to the per-metric adjustments to mitigate collinearity and avoid double-counting shared physiological effects before forming the total age delta.

Illustratively, a 34.5-year-old male might have per-metric adjustments such as: VO₂ max −3.6 years; resting heart rate −0.8 years; BMI −2.5 years; sleep duration +0.6 years; Zone 2/3 time+0.1 years; Zone 4/5 time 0 years; and sedentary time+1.6 years. These contributions may be summed to yield a total age delta of −4.6 years, which may be applied to the chronological age (34.5-4.6) to produce an effective age of 29.9 years. This calculation may be performed on a daily, weekly, or monthly basis, and may be used to update age-related data for the user, which may be displayed in a user interface such as any of the user interfaces described herein.

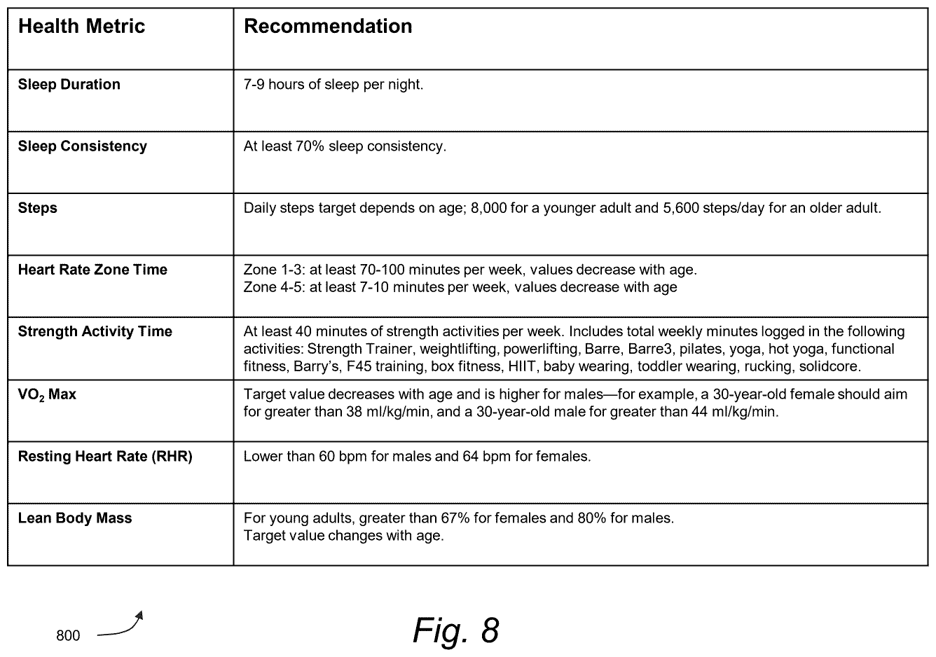

FIG. 10 illustrates age adjustments for sleep consistency. In order to obtain age adjustments, sleep consistency data from a physiological monitor may first be aligned to a sleep regularity index (SRI) or similar metric used in all-cause-mortality studies. For example, a wearable (or associated processing resource) may compute a probability (0-100%) that the user is in the same state—awake or asleep—at matching clock times 24 hours apart over several days. This figure of merit may then be calibrated to published SRI percentiles (e.g., bottom 20%, middle 20%, top 20%) so that a given wearable-derived consistency value can be mapped to exposure categories in a correspondingly reported all-cause mortality study. Once this mapping is established, each sleep-consistency band may be assigned a hazard ratio relative to a chosen reference and these bands can be transformed into age adjustments as described herein. It will also be appreciated that the age adjustments may be scaled or otherwise adjusted based on user demographics, such as chronological age and gender. As described herein, a user's chronological age may be adjusted according to the age adjustments for sleep consistency to provide an effective age for the user based on sleep consistency data and the correlated age adjustments illustrated in FIG. 10.

In general, age adjustments for sleep consistency may be implemented as a lookup table, e.g., as illustrated in FIG. 10, as a machine learning model trained on a corresponding data set for sleep consistency and all-cause mortality (or other risk measurement or the like), as a linear regression, or any other model or the like that converts sleep consistency data into a continuous or binned adjustment to effective age in a way that reflects changes in the hazard ratio for an individual.

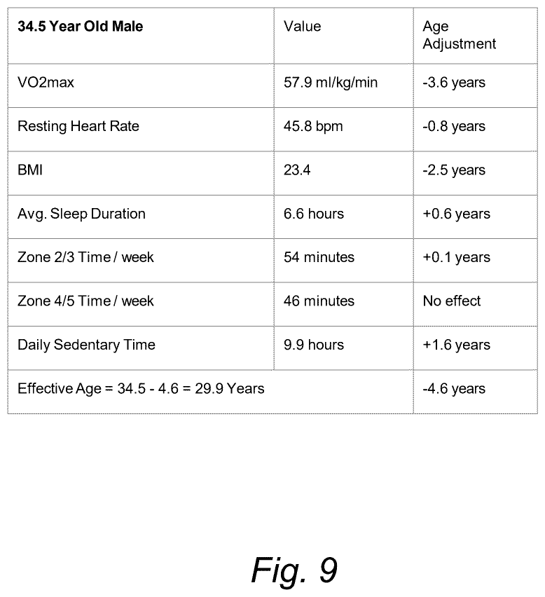

FIG. 11 illustrates age adjustments for strength training. In one aspect, strength-training data can be adapted to an age adjustment by mapping weekly strength time to hazard ratios from all-cause mortality studies and then converting those hazard ratios into “years” of age advancement. A user's weekly strength-training time (for example, minutes per week logged in resistance activities) may first be averaged over a longer window (e.g., 180 days) to obtain a stable exposure level. This exposure may then be positioned on a dose-response curve derived from cohort studies that report all-cause mortality hazard ratios as a function of muscle-strengthening time. For example, one meta-analysis indicated an optimally effective range of about 30-60 minutes of strength training per week, with a minimum at zero minutes and a plateau for beneficial effect at 120 minutes. Using the associated curve, which is typically J-shaped or U-shaped rather than linear, a hazard ratio can be assigned to observed weekly minutes, optionally capping the benefit beyond a chosen upper bound (for instance, ignoring time beyond about 2 hours per week to reflect the U-shaped relationship seen in some studies). Once the hazard ratio is characterized in this manner, it may be converted into effective age adjustments as described herein.

In general, age adjustments for strength training may be implemented as a lookup table, e.g., as illustrated in FIG. 11, as a machine learning model trained on a corresponding data set for strength training and all-cause mortality (or other risk measurement or the like), as a linear regression, or any other model or the like that converts strength training data into a continuous or binned adjustment to effective age in a way that reflects changes in the hazard ratio for an individual. It will also be appreciated that the age adjustments may be scaled or otherwise adjusted based on user demographics, such as chronological age and gender. As described herein, a user's chronological age may be adjusted according to strength training to provide an effective age for the user based on strength training data and the correlated age adjustments illustrated in FIG. 11.

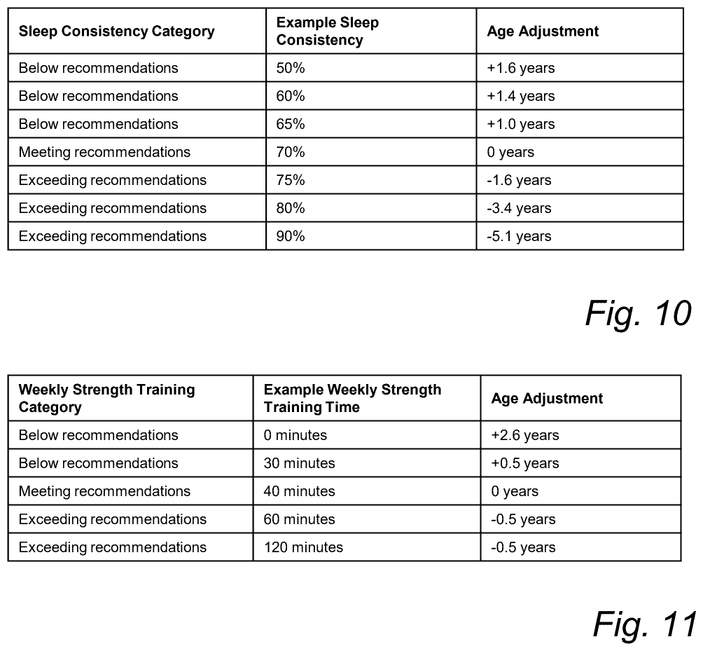

FIG. 12 illustrates age adjustments for lean body mass (percentage). Because lean body mass distributions differ markedly by sex and age, a user's lean body mass percentage may be located on an age- and sex-specific reference curve derived from cohort studies that report all-cause mortality as a function of body fat or lean mass (for example, percentile curves from DXA-based studies). A “healthy” referent band—such as approximately the 70th-90th percentile of lean mass for a given age/sex group—may be selected so that values within or above that band correspond to a hazard ratio at or below 1.0, while values below the lower bound of the band correspond to hazard ratios that increase as lean mass declines. For example, lean mass below about 60% for a 30-year-old woman, or below about 78% for a 30-year-old man, may map to progressively higher hazard ratios based on the reported dose-response relationship between body fat percentage and all-cause mortality. Once a specific hazard ratio is assigned to the user's lean mass percentage, this can be converted into an effective age adjustment as described herein.

In general, age adjustments for lean mass may be implemented as a lookup table, e.g., as illustrated in FIG. 12, as a machine learning model trained on a corresponding data set for lean mass and all-cause mortality (or other risk measurement or the like), as a linear regression, or any other model or the like that converts lean mass data into a continuous or binned adjustment to effective age in a way that reflects changes in the hazard ratio for an individual. It will also be appreciated that the age adjustments may be scaled or otherwise adjusted based on user demographics such as chronological age and gender. As more specifically illustrated in FIG. 12, the age adjustments may depend specifically on gender and age, with a 30-year change in chronological age yielding different age adjustments over a range of lean mass percentages.

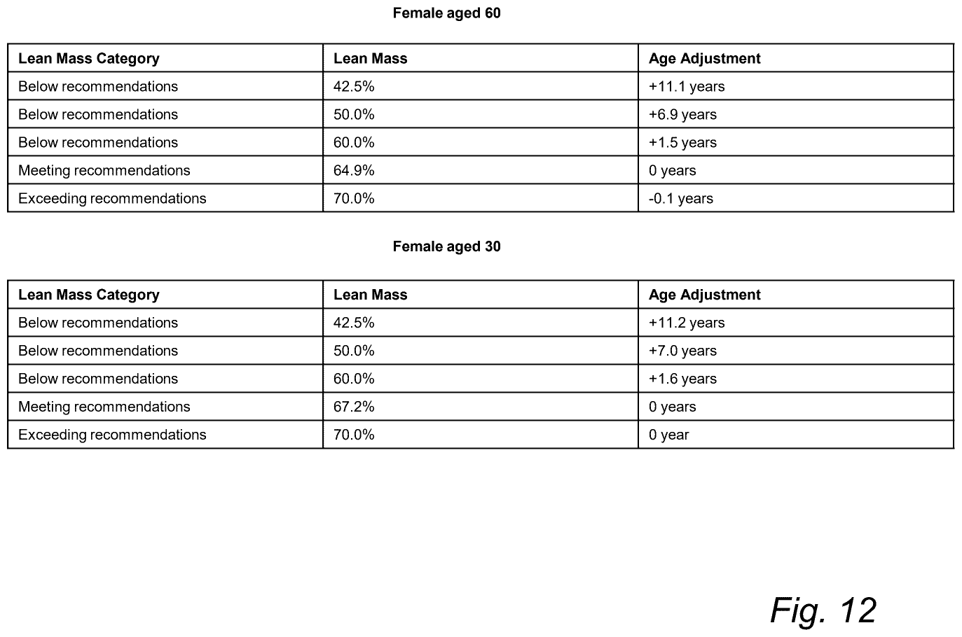

FIG. 13 illustrates age adjustments for VO₂ max. In general, a user's VO₂ max (for example, a six-month average of wearable-derived estimates) may be compared to an age- and sex-specific referent VO₂ max associated with preserved function for independent living (e.g., 20 ml·kg⁻¹·min⁻¹) at advanced ages or with the lowest observed all-cause mortality in cohort data. The user's percentile or absolute distance from this referent may then be mapped to a hazard ratio using dose-response relationships from prospective studies (for example, one study shows each 1-MET increase ≈3.5 ml·kg⁻¹·min⁻¹ corresponding to a ≈0.87-0.90 hazard). Once a VO₂-max-specific hazard ratio is obtained in this manner, it can be converted into an effective age adjustment as described herein.

In general, age adjustments for VO₂ max may be implemented as a lookup table, e.g., as illustrated in FIG. 13, as a machine learning model trained on a corresponding data set for VO₂ max and all-cause mortality (or other risk measurement or the like), as a linear regression, or any other model or the like that converts VO₂ max data into a continuous or binned adjustment to effective age in a way that reflects changes in the hazard ratio for an individual. It will also be appreciated that the age adjustments may be scaled or otherwise adjusted based on user demographics such as chronological age and gender. As described herein, a user's chronological age may be adjusted according to VO₂ max to provide an effective age for the user based on VO₂ max data and a correlated age adjustment.

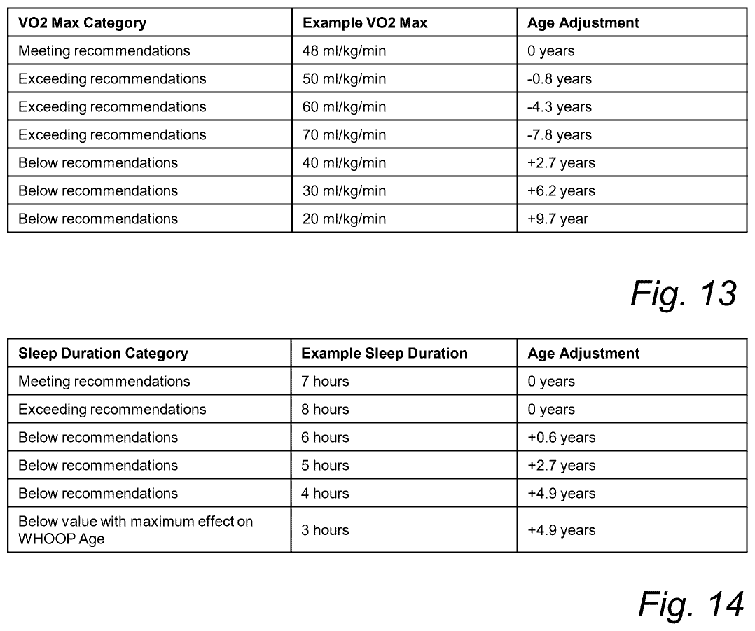

FIG. 14 illustrates age adjustments for sleep duration. In an aspect, sleep duration may be measured as the average nightly total sleep time over a long window—e.g., a trailing one-hundred-eighty-day mean derived from wearable-detected sleep onset and wake times. This average may then be compared to a guideline-based referent such as at least about seven hours per night, with values below the referent treated as “short sleep” and values at or above the referent treated as “adequate sleep.” Based on all-cause-mortality literature (for example, meta-analyses reporting hazard ratios for categories such as <5 h, 5-6 h, 6-7 h, and 7-9 h), each duration band may be assigned a hazard ratio relative to the referent band (e.g., 7-9 h). Once sleep-duration hazard ratios are characterized in this manner, they can be converted into an adjustment to effective age using the techniques described herein.

In general, age adjustments for sleep duration may be implemented as a lookup table, e.g., as illustrated in FIG. 14, as a machine learning model trained on a corresponding data set for strength training and all-cause mortality (or other risk measurement or the like), as a linear regression, or any other model or the like that converts sleep duration data into a continuous or binned adjustment to effective age in a way that reflects changes in the hazard ratio for an individual. Sleep duration presents a health metric where the current literature does not appear to support differentiation based on age or gender. As such, a single model may be used for all demographics. As described herein, a user's chronological age may be adjusted according to sleep duration to provide an effective age for the user based on sleep duration data and the correlated age adjustments illustrated in FIG. 14.

The foregoing examples illustrate the manner in which particular health metrics can be converted into age adjustments over a range of values, and how these adjustments can vary based on other factors such as chronological age or gender. In general, any health metric for which mortality data supports the determination of a hazard ratio relative to a suitable referent group can be used as a health metric to support adjustments to an effective age as described herein. More generally, an adjustment to a user's effective age can be based on any health metric where scientific literature supports a relationship with all-cause mortality, preferably where the association cannot be mostly explained by other included metrics and there is a plausible pathway connecting the metric to long-term health.

In this context, the referent group selection can be foundational to effective age calculations because the referent group defines the baseline risk against which measured behaviors and other health metrics are compared, thereby controlling the sign, magnitude, and stability of the resulting age adjustments. Choosing guideline-based referents (for example, based on public health and physician recommendations such as 150 minutes of weekly exercise, 7-9 hours of sleep, age-appropriate and sex-appropriate VO₂ max and lean mass thresholds, and resting heart rate near lower clinical percentiles) can help to anchor age adjustment models to clinically meaningful targets: failing to meet the referent yields hazard ratios greater than one and positive age advancements, while meeting or exceeding the referent yields hazard ratios at or below one and non-positive advancements. By contrast, cohort averages (e.g., an “average user” or population mean) can unintentionally promote suboptimal behaviors, compress dynamic range, and weaken coaching signals, particularly in cases where users might receive neutral or favorable adjustments despite not meeting recommended levels.

Referent choice may also affect calibration across demographics. Age-specific and sex-specific referents for VO₂ max or lean mass can maintain a zero point that is physiologically appropriate. Stability can also be supported by guideline-based referents, which change slowly and support consistent year-over-year tracking. By contrast, cohort-based referents can drift with seasonality, membership mix, or geography, introducing spurious swings in effective age unrelated to a user's true health trajectory. Finally, once a referent is fixed, metric exposures can be mapped to all-cause mortality hazard ratios and converted to age deltas in a manner such that the referent directly sets the zero for age adjustments, as well as how much headroom exists for negative and positive adjustments. Thus, a carefully selected, clinically grounded referent can support reliable estimates of the impact of behaviors on health and wellness, thus supporting improved trustworthiness, coaching, and user experience.

A suitable choice of referents for modeling of age adjustments as described herein provides a useful framework for expressing the impact of various behaviors and observations with a result—effective age—that facilitates intuitive user understanding and ease of coaching. However, the concurrent use of multiple health metrics can also present challenges. In particular, hazard ratios for individual health metrics are based on findings from studies that might not be controlled for all the health metrics in an effective age calculation. This means that health metrics that are correlated to one another, or that are derived from shared underlying data (e.g., cardiac data and motion data from a wearable monitor) can overstate the impact of a contributing measurement. For example, where sleep consistency is correlated to sleep duration, an adjustment for sleep consistency might be attributable in part to sleep duration, i.e., the user is benefiting from more sleep, not just higher sleep consistency. Although both metrics might sometimes change in a correlated fashion, the corresponding effect on risk should not be doubled as a result.

In one aspect, a two-stage weighting strategy may be used to address this issue and eliminate double counting of the impact of several health metrics that share the same underlying physiology or are otherwise correlated to one another. In a first stage, the unadjusted contributions may be calculated. Each health metric yields an unadjusted age adjustment AA from its own hazard-ratio mapping applied to the population data, as described herein. In a second stage, weights may be derived for each age adjustment. Using a large, representative sample of user data, the vector of unadjusted component deltas:

X=[ΔA₁,ΔA₂, . . . ,ΔAₙ]

can be regressed onto the unadjusted total delta:

y=ΣᵢΔAᵢ.

The resulting coefficients β estimate the independent contribution of every metric after accounting for covariance among metrics:

β=(X^{T}X)⁻¹X^{T}y

Because correlated predictors inflate X^{T}X, the corresponding βᵢ shrink toward zero, automatically down-weighting overlapping signals. To find the final effective age adjustment, the adjusted contribution of each metric becomes βᵢ ΔAᵢ, and the total age adjustment is the weighted sum

$$Δ {A}_{total} = {\sum }_{i = 1}^{n} {β}_{i} Δ {A}_{i}$$

This single ΔAₜₒₜₐₗ can then be added to (or subtracted from) chronological age to obtain the user's effective age, while the β-weights ensure that overlapping physiological effects are counted only once.

In another aspect, a structural equation model may be used to correct for the overlapping effects of different data sources and/or health metrics. In general, structural equation modeling may be used to correct for interdependencies among health metrics by explicitly modeling both their shared latent structure and their direct contributions to an effective-age outcome. In an aspect, a structural model may be constructed in which observed health metrics (for example, sleep duration, sleep consistency, VO₂ max, resting heart rate, lean mass, daily steps, heart-rate zone minutes, and sedentary time) are treated as indicators of one or more latent variables that represent underlying physiological domains such as “cardiorespiratory fitness,” “sleep regularity,” or “metabolic health.” The model may specify both direct paths from each latent factor to an effective-age latent variable (or to an all-cause-mortality risk latent variable that is later transformed into age), and indirect paths among observed metrics (e.g., sleep consistency influencing sleep duration, which in turn influences resting heart rate). Using a large empirical covariance matrix of unadjusted metric values Σ_{empirical}, the structural model may be estimated to produce a theoretical covariance matrix Σ_{model}(θ), where θ denotes factor loadings and path coefficients. Estimation may proceed by minimizing a discrepancy function such as:

F(θ)=∥Σ_{empirical}−Σ_{model}(θ)|

where the discrepancy function is subject to identification constraints on latent variables. The resulting factor loadings and path coefficients may then be used to decompose each observed metric's contribution into unique (direct) and shared (indirect) components. For effective-age calculations as described herein, only the unique (or de-overlapped) component of each metric's association with the effective-age latent variable may be retained when deriving per-metric age adjustments, while the overlapping variance explained by other metrics and shared latent factors can be discounted. In this manner, structural equation modeling provides a technique for handling collinearity, preventing double counting of shared physiological effects, and ensuring that the aggregate effective-age estimate reflects the independent contribution of each health metric.

In one implementation, a large data set of (unadjusted) health metric data from users of wearable physiological monitors was used to compute an empirical covariance matrix capturing correlations between health metrics. A theoretical covariance matrix was then derived, representing how these components would behave if their effects were independent. The difference between these two matrices was minimized, yielding adjustment factors to correct for interdependencies among health metrics. These adjustment factors were then applied to the hazard ratios of each health metric, ensuring that an effective age calculated from a combination of health metrics reflected the independent contribution of each behavior without artificial inflation from correlated metrics. As a result, an age adjustment calculated in this manner more accurately reflects a user's all-cause mortality risk based on all of the measured health metrics used to calculate the component age adjustments.

More generally, these approaches prevent double-counting effects to provide a more accurate assessment of long-term risk, thus helping effective age calculations remain grounded in available health data and research, while being personalized to each individual's objectively measurable health profile. Thus, in one aspect, where a common set of the data from the physiological monitor is used to derive two or more of the health metrics, or where two or more of the health metrics are otherwise correlated, a plurality of weights for the weighted sum of the plurality of age adjustments may be derived to mitigate double counting of the age impact of the data among the plurality of age adjustments derived for the plurality of health metrics. The weights may be statistically derived by regressing a vector of unadjusted component deltas of individual age adjustments onto the unadjusted total delta for effective age. The weights may also or instead be statistically derived with a structural equation model that corrects for the overlapping effects of different data sources and/or health metrics.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors, or other programmable devices or processing circuitry, along with internal and/or external memory. This may also, or instead, include one or more application-specific integrated circuits, programmable gate arrays, programmable array logic components, or any other device or devices that may be configured to process electronic signals. It will further be appreciated that a realization of the processes or devices described above may include computer-executable code created using a structured programming language such as C, an object-oriented programming language such as C++, or any other high-level or low-level programming language (including assembly languages, hardware description languages, and database programming languages and technologies) that may be stored, compiled, or interpreted to run on one of the above devices, as well as heterogeneous combinations of processors, processor architectures, or combinations of different hardware and software.

Thus, in one aspect, each method described above, and combinations thereof, may be implemented as a computer program product comprising computer-executable code embodied in a non-transitory computer-readable medium (such as any of the memories described herein) that, when executing on one or more computing devices, performs the corresponding steps thereof. In another aspect, the methods may be embodied in systems that perform the steps thereof and may be distributed across devices in a number of ways, or all of the functionalities may be integrated into a dedicated, standalone device or other hardware. The code may be stored in a non-transitory fashion in a computer memory, which may be a memory from which the program executes (such as random access memory associated with a processor), or a storage device such as a disk drive, flash memory, or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party, such as a remote user, a remote processing resource (e.g., a server or cloud computer), or a machine, to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

1. A computer program product comprising computer-executable code embodied in a non-transitory computer-readable medium that, when executing on one or more computing devices, causes the one or more computing devices to perform the steps of:
acquiring data from a physiological monitor worn by a user, the data including cardiac activity data for the user and movement data for the user;
calculating a plurality of metrics for the user based on the data from the physiological monitor, the plurality of metrics including at least:
a resting heart rate for the user based on the cardiac activity data;
an exercise metric for the user based on the cardiac activity data recorded during an exercise activity,
a sleep metric derived from the data for the user, and
a movement metric for the user derived from the movement data for the user;
calculating a first plurality of age adjustments based on the plurality of metrics;
acquiring a plurality of health data measurements for the user including at least:
a chronological age of the user,
a body mass index of the user, and
a maximal oxygen consumption for the user;
calculating a second plurality of age adjustments based on the plurality of health data measurements; and
calculating an effective age of the user by adjusting the chronological age of the user according to each of the first plurality of age adjustments and each of the second plurality of age adjustments.

2. The computer program product of claim 1, further comprising computer-executable code that causes the one or more computing devices to perform the step of displaying the effective age of the user on a user device.

3. The computer program product of claim 1, further comprising computer-executable code that causes the one or more computing devices to perform the step of displaying the effective age, the chronological age, and a pace of aging of the user based on a difference between the chronological age and the effective age.

4. The computer program product of claim 1, wherein the maximal oxygen consumption for the user is estimated based on the data from the physiological monitor.

5. The computer program product of claim 1, wherein the resting heart rate is measured during an automatically detected sleep interval for the user.

6. The computer program product of claim 1, wherein the sleep metric includes one or more of a quantity of sleep, a sleep debt, and a sleep consistency.

7. The computer program product of claim 1, wherein the plurality of metrics include a movement metric based on detecting steps by the user with the movement data.

8. The computer program product of claim 1, wherein the movement metric is based on one or more of strength training repetitions, a daily step count for the user, a timing of sedentary and active intervals, a distance traveled during physical activity by the user, and an activity categorization based on the movement data.

9. The computer program product of claim 1, wherein the exercise metric includes an average daily time within one or more target heart rate zones.

10. The computer program product of claim 1, wherein one or more of the plurality of metrics are averaged over at least seven days.

11. A method comprising:
acquiring data for a user from a physiological monitor worn by the user;
calculating a plurality of health metrics for the user from the data;
calculating a plurality of age adjustments for the user based on the plurality of health metrics, wherein each of the plurality of age adjustments is derived from a hazard ratio for a corresponding health metric based on an all-cause mortality rate for a population of users;
calculating a total age adjustment for the user based on a weighted sum of the plurality of age adjustments; and
calculating an effective age for the user by adjusting a chronological age of the user according to the total age adjustment.

12. The method of claim 11, further comprising displaying the effective age to the user.

13. The method of claim 11, wherein the data for the user includes at least one of cardiac data and movement data.

14. The method of claim 11, wherein the plurality of health metrics include one or more of a resting heart rate for the user, a heart rate variability for the user, a daily step count for the user, a strength metric, and a maximal oxygen consumption for the user.

15. The method of claim 11, wherein the plurality of health metrics include a sleep metric measuring at least one of a quantity of sleep, a quality of sleep, a quantity of restorative sleep, and a consistency of sleep.

16. The method of claim 11, wherein the plurality of health metrics include a strain score based on heart rate zones for one or more exercise activities by the user.

17. The method of claim 11, wherein a common set of the data from the physiological monitor is used to derive two or more of the plurality of health metrics, and wherein a plurality of weights for the weighted sum of the plurality of age adjustments are statistically derived to mitigate double counting of an age impact of the data among the plurality of age adjustments derived for the plurality of health metrics.

18. A system comprising:
a physiological monitor configured to acquire data for a user;
one or more processors configured by computer-executable code stored in a memory to perform the steps of:
receiving the data from the physiological monitor,
calculating a plurality of health metrics for the user from the data,
calculating a plurality of age adjustments for the user based on the plurality of health metrics, wherein each of the plurality of age adjustments is derived from a hazard ratio for a corresponding health metric based on an all-cause mortality rate for a population of users,
calculating a total age adjustment for the user based on a weighted sum of the plurality of age adjustments, and
calculating an effective age for the user by adjusting a chronological age of the user according to the total age adjustment; and
a user device configured to display the effective age to the user.

19. The system of claim 18, wherein the one or more processors include processing resources on the user device.

20. The system of claim 18, wherein the one or more processors include processing resources of a remote server coupled in a communicating relationship with the physiological monitor and the user device.

21-46. (canceled)
